(19) United States
(12) Patent Application Publication
(10) Pub. No.: US 2007/0066570 A1
(43) Pub. Date: Mar. 22, 2007

(54) Methods for treating sleep disorders

(76) Inventor: Michael Solomon, Concord, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/560,557

(22) PCT Filed: Jun. 16, 2004
(86) PCT No.: PCT/US04/18985

§ 371 Date: Apr. 28, 2006

(30) Foreign Application Priority Data
Jun. 16, 2003 (US) 60478764
Aug. 20, 2003 (US) 60496621

(51) Int. Cl. A61K 31/675; A61K 31/445; A61K 31/454; A61K 31/4245; A61K 31/41; A61K 31/40

(52) U.S. Cl. 514/89; 514/319; 514/364; 514/381; 514/408; 514/91

(57) ABSTRACT

A method of treating a subject for a sleeping disorder, comprising administering to a subject in need of treatment for a sleeping disorder an effective amount of an N-substituted azaheterocyclic compound as described herein.

### BACKGROUND OF THE INVENTION

Sleep disorders arising from difficulty in falling asleep or remaining asleep are a significant medical issue, resulting in problem sleepiness that can impair health and safety for those affected. Such disorders can arise from endogenous conditions, e.g., sleep apnea, insomnia, and the like, or from external stresses, e.g., the disruptive effect of shift work schedules or “jet lag” on normal sleep patterns.

Existing pharmaceutical treatments for aiding sleep include sedatives or hypnotics, e.g., benzodiazepine or barbiturate derivatives, and milder agents, e.g., antihistamines. These treatments are known to have numerous drawbacks, including loss of effect after long-term use, addictive potential, abuse potential, persistence of unwanted sedative effects past the desired sleep period, side effects due to nonspecific activity, and the like.

Therefore, there is a need for improved pharmaceutical treatments for aiding sleep in subjects in need of such treatment.

### SUMMARY OF THE INVENTION

The invention is based in part on the discovery that certain N-substituted azaheterocyclic compounds are effective as sleeping aids. Methods of treating sleeping disorders with such compounds are provided herein.

The method is used, for example, to treat sleep disorders including circadian rhythm abnormality, insomnia, parasomnia, sleep apnea syndrome, narcolepsy and hypersomnia. In general, in one aspect, the method is used to treat circadian rhythm abnormalities including jet lag, shift-work disorders, delayed sleep phase syndrome, advanced sleep phase syndrome and non-24 hour sleep-wake disorder. In another aspect, the method is used to treat insomnia including extrinsic insomnia, psychophysiologic insomnia, altitude insomnia, restless leg syndrome, periodic limb movement disorder, medication-dependent insomnia, drug-dependent insomnia, alcohol-dependent insomnia and insomnia associated with mental disorders.

In general, in another aspect, the method treats parasomnias including somnambulism, pavor nocturnus, REM sleep behavior disorder, sleep bruxism and sleep enuresis. In yet another aspect, the method is used to treat sleep apnea disorder including central sleep apnea, obstructive sleep apnea and mixed sleep apnea. Additionally, the method is used to treat other sleep disorders such as narcolepsy or hypersomnia.

One embodiment of the invention is a method of treating a subject for a sleeping disorder, comprising administering to a subject in need of treatment for a sleeping disorder an effective amount of a compound represented by structural formula I, or a pharmaceutically acceptable salt, solvate, or hydrate thereof:

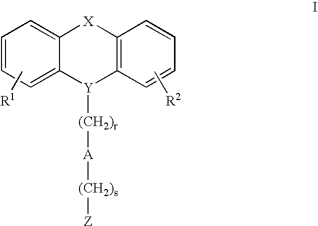

The variables r and s are independently 0, 1, 2, 3 or 4.

R¹ and R² independently are —H, halogen, hydroxy, —CN, —NO₂, optionally halogenated C₁₋₆-alkyl or C₁₋₆-alkoxy, —NR²¹R¹², —(SO₂)NR²¹R²², —NR²¹(SO₂)NR²², —(CO)NR²¹R²², —NR²¹(CO)R²², —(CO)R²², or —(CO₂)R²², and R²¹ and R²² independently are —H or C₁₋₆-alkyl.

X is —O—, —S—, —O—CH₂—, —CH₂—O—, —O—CH₂—O—, —CH₂—O—CH₂—, —S—CH₂—, —CH₂—S—, —CH₂CH₂CH₂—, —CH₂CH₂—, —C(R³R⁴)—, —CH(R⁶)CH₂—, —CH₂CH(R⁶)—, —CH═CH—CH₂—, —CH₂—CH═CH—, —CH═CH—, ortho-phenylene, —(C═O)—, —(S═O)—, —CH₂—(C═O)—, —(C═O)—CH₂—, —N(R⁵)—(C═O)—, —(C═O)—N(R⁵)—, —N(CH₃)SO₂—, —SO₂N(CH₃)—, —(CH₂)N(R⁵)—, —N(R⁵)(CH₂)—, or —N(R⁷)—, R³, R⁴, R⁵, R⁶, and R⁷ independently are —H, phenyl, or C₁₋₆-alkyl.

Y is >N—, >CH—, >N—(C═O)— or >C═C(R⁸)—. R⁵ is —H, phenyl, or C₁₋₆-alkyl. In Y, only the underscored atom is a ring atom.

A is —CH═CR⁹—, —CR⁹═CH—, —C≡C—, phenylene, C₃₋₇ -cycloalkylene, the completion of a bond, —(C═O)—, —(C═CH₂)—, —(CR⁹R¹⁰)—, —CH(OR¹¹)—, or —CH(NHR¹¹)—. R⁹ and R¹⁰ independently are —H, C₁₋₆-unbranched alkyl, C₃₋₆ -branched allyl or C₃₋₇-cycloalkyl. R¹¹ is —H, optionally halogenated C₁₋₆-alkyl or C₁₋₆-alkoxy, or phenyl optionally substituted with halogen, hydroxy, or optionally halogenated C₁₋₆-alkyl or C₁₋₆-alkoxy.

Z is an optionally substituted group selected from a carboxylic acid or bioisostere thereof; an ester, a nonaromatic 3-7 membered nitrogen-containing heterocycle that is optionally fused to an aryl ring; a secondary or tertiary amine substituted with an aryl, aralkyl, heteroaryl, heteroaralkyl, 3-7 membered nonaromatic heterocycle, or 3-7 membered cycloalkyl group; or —N(R¹¹)BR¹². B is C₁₋₆-alkylene, C₂₋₆-alkenylene or C₂₋₆-alkynylene. R¹² is —(CH₂)ₘOH or —(CH₂)ₚCOR¹⁷, m is 0, 1, 2, 3, 4, 5 or 6, and p is 0 or 1. R¹⁷ is —OH, —NHR²⁰ or C₁₋₆-alkoxy and R²⁰ is —H or C₁₋₆-alkyl.

One embodiment of the invention is a method of treating a subject for a sleeping disorder, comprising administering to a subject in need of treatment for a sleeping disorder an effective amount of a compound represented by structural formula II, or a pharmaceutically acceptable salt, solvate, or hydrate thereof.

Another embodiment of the invention is a method of treating a subject for a sleeping disorder, comprising administering to a subject in need of treatment for a sleeping disorder an effective amount of a compound represented by structural formula IIIa, or a pharmaceutically acceptable salt, solvate, or hydrate thereof.

Another embodiment of the invention is a method of treating a subject for a sleeping disorder, comprising administering to a subject in need of treatment for a sleeping disorder an effective amount of a compound represented by structural formula IIIb, or a pharmaceutically acceptable salt, solvate, or hydrate thereof.

Another embodiment of the invention is a method of treating a subject for a sleeping disorder, comprising administering to a subject in need of treatment for a sleeping disorder an effective amount of a compound represented by structural formula IVa, or a pharmaceutically acceptable salt, solvate, or hydrate thereof.

Another embodiment of the invention is a method of treating a subject for a sleeping disorder, comprising administering to a subject in need of treatment for a sleeping disorder an effective amount of a compound represented by structural formula Vb, or a pharmaceutically acceptable salt, solvate, or hydrate thereof.

Another embodiment of the invention is a method of treating a subject for a sleeping disorder, comprising administering to a subject in need of treatment for a sleeping disorder an effective amount of a compound represented by structural formula Va, or a pharmaceutically acceptable salt, solvate, or hydrate thereof.

Another embodiment of the invention is a method of treating a subject for a sleeping disorder, comprising administering to a subject in need of treatment for a sleeping disorder an effective amount of a compound represented by structural formula Vb, or a pharmaceutically acceptable salt, solvate, or hydrate thereof.

Another embodiment of the invention is a method of treating a subject for a sleeping disorder, comprising administering to a subject in need of treatment for a sleeping disorder an effective amount of a compound 1, or a pharmaceutically acceptable salt, solvate, or hydrate thereof.

Another embodiment of the invention is a method of treating a subject for a sleeping disorder, comprising administering to a subject in need of treatment for a sleeping disorder an effective amount of a compound 1-R, or a pharmaceutically acceptable salt, solvate, or hydrate thereof.

Another embodiment of the invention is a method of treating a subject for a sleeping disorder, comprising administering to a subject in need of treatment for a sleeping disorder an effective amount of a compound 1-S, or a pharmaceutically acceptable salt, solvate, or hydrate thereof.

In another aspect, the invention involves modulating sleep by administering a therapeutically effective amount of a compound of the invention, or a pharmaceutically effective salt thereof, to a subject. The method modulates sleep several ways including decreasing the time to sleep onset, increasing the average sleep bout length, or increasing the maximum sleep bout length.

The invention is useful for treating subjects that have a sleep disorder, in particular subjects that have difficulty falling asleep or remaining asleep. The invention provides improved sleep aid while also providing fewer side effects compared to existing sedatives.

The method includes the use of a pharmaceutically acceptable salt of a compound of the invention in the treatment of a sleep disorder. In one embodiment, the compound, or a pharmaceutically acceptable salt thereof, is administered as a monotherapy or co-administered with one or more additional agents as a co-therapy. The compound of the invention is administered orally, nasally, transdermally, pulmonarily, inhalationally, buccally, sublingually, intraperintoneally, intravenously, rectally, intrapleurally, intrathecally or parenterally. In one particular embodiment, the compound of the invention or pharmaceutically acceptable salt thereof is administered orally. The compound of the invention or pharmaceutically acceptable salt thereof is included in a pharmaceutical composition and is administered to a subject, including a human subject. Other subjects include farm animals, companion animals, laboratory animals and wild animals.

The above description sets forth rather broadly the more important features of the present invention in order that the detailed description thereof that follows may be understood, and in order that the present contributions to the art may be better appreciated. Other objects and features of the present invention will become apparent from the following detailed description considered in conjunction with the examples.

### BRIEF DESCRIPTION OF THE DRAWINGS

FIG. 1 is a graph depicting non-rapid eye movement (NREM) sleep cumulation, in minutes, versus time (in hours) after administration of compound 1 (racemate) at 10 mg/kg (grey line) and 30 mg/kg (black line) versus vehicle control (grey band).

FIG. 2 is a graph depicting NREM sleep cumulation, in minutes, versus time (in hours) after administration of compound 1-R at 10 mg/kg (grey line) and 30 mg/kg (black line) versus vehicle control (grey band).

FIG. 3 is a graph depicting NREM sleep cumulation, in minutes, versus time (in hours) after administration of compound 1-S at 10 mg/kg (grey line) and 30 mg/kg (black line) versus vehicle control (grey band).

FIG. 4 is a graph depicting longest uninterrupted sleep bout (LUSB) for each hour of circadian time after administration of compound 1 (racemate) at 30 mg/kg (black line) versus vehicle control (grey band).

FIG. 5 is a graph depicting LUSB for each hour of circadian time after administration of compound 1-R at 30 mg/kg (black line) versus vehicle control (grey band).

FIG. 6 is a graph depicting LUSB for each hour of circadian time after administration of compound 1-S at 30 mg/kg (black line) versus vehicle control (grey band).

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present specification will control.

The invention relates, in part, to methods of treating sleep disorders using N-substituted azaheterocyclic compounds. In particular, the invention treating subjects that have difficulty falling or remaining asleep. A description of preferred embodiments of the invention follows.

One embodiment of the invention is a method of treating a subject for a sleeping disorder, comprising administering to a subject in need of treatment for a sleeping disorder an effective amount of a compound represented by structural formula I, or a pharmaceutically acceptable salt, solvate, or hydrate thereof:

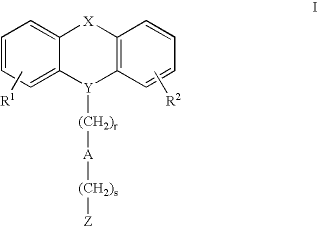

The variables r and s are independently 0, 1, 2, 3 or 4.

R¹ and R² independently are —H, halogen, hydroxy, —CN, —NO₂, optionally halogenated C₁₋₆-alkyl or C₁₋₆-alkoxy, —NR²¹R²², —(SO₂)NR²¹R²², —NR²¹(SO₂)NR²², —(CO)NR²¹R²², —NR²¹(CO)R²², —(CO)R²², or —(CO₂)R²², and R²¹ and R²² independently are —H or C₁₋₆alkyl.

X is —O—, —S—, —O—CH₂—, —CH₂—O—, —O—CH₂—O—, —CH₂—O—CH₂—, —S—CH₂—, —CH₂—S—, —CH₂CH₂CH₂—, —CH₂CH₂—, —C(R³R⁴)—, —CH(R⁶)CH₂—, —CH₂CH(R⁶)—, —CH═CH—CH₂—, —CH₂—CH═CH—, —CH═CH—, ortho-phenylene, —(C═O)—, —(S═O)—, —CH₂—(C═O)—, —(C═O)—CH₂—, —N(R⁵)—(C═O)—, —(C═O)—N(R⁵)—, —N(CH₃)SO₂—, —SO₂N(CH₃)—, —(CH₂)N(R⁵)—, —N(R⁵)(CH₂)—, or —N(R⁷)—. R³, R⁴, R⁵, R⁶, and R⁷ independently are —H, phenyl, or C₁₋₆-alkyl.

Y is >N—, >CH—, >N—(C═O)— or >C═C(R⁸)—. R⁸ is —H, phenyl, or C₁₋₆-alkyl. In Y, only the underscored atom is a ring atom.

A is —CH═CR⁹—, —CR⁹═CH—, —C≡C—, phenylene, C₃₋₇ -cycloalkylene, the completion of a bond, —(C═O)—, —(C═CH₂)—, —(CR⁹R¹⁰)—, —CH(OR¹¹)—, or —CH(NHR¹¹)—. R⁹ and R¹⁰ independently are —H, C₁₋₆-unbranched alkyl, C₃₋₆ -branched alkyl or C₃₋₇-cycloalkyl. R¹¹ is —H, optionally halogenated C₁₋₆-alkyl or C₁₋₆-alkoxy, or phenyl optionally substituted with halogen, hydroxy, or optionally halogenated C₁₋₆-alkyl or C₁₋₆-alkoxy.

Z is an optionally substituted group selected from a carboxylic acid or bioisostere thereof; an ester, a nonaromatic 3-7 membered nitrogen-containing heterocycle that is optionally fused to an aryl ring; a secondary or tertiary amine substituted with an aryl, aralkyl, heteroaryl, heteroaralkyl, 3-7 membered nonaromatic heterocycle, or 3-7 membered cycloalkyl group; or —N(R¹¹)BR¹². B is C₁₋₆-alkylene, C₂₋₆-alkenylene or C₂₋₆-alkynylene. R¹² is —(CH₂)ₘOH or —(CH₂)ₚCOR¹⁷, m is 0, 1, 2, 3, 4, 5 or 6, and p is 0 or 1. R¹⁷ is —OH, —NHR²⁰ or C₁₋₆alkoxy and R²⁰ is —H or C₁₋₆-alkyl.

In a preferred embodiment, the compound employed in the disclosed methods is represented by structural formula I′:

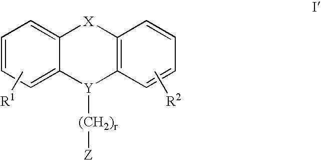

The variables in structural formula I′ are as provided in structural formula I.

In a preferred embodiment, R¹ and R² independently are —H, halogen, hydroxy, —CN, —NO₂, optionally halogenated C₁₋₆-alkyl or C₁₋₆-alkoxy, —NHR²², —(SO₂)NHR²², —NH(SO₂)R²², —(CO)NHR²², —NH(CO)R²², —(CO)R²², or —(CO₂)R²². Even more preferably, R¹ and R² are selected from —H, —F, —Cl, —Br, —CF₃, —OCF₃, or C₁₋₆-alkyl. Preferably R¹ and R² are —H, —Cl or methyl. Preferably, at least one of R¹ and R² is —H. Most preferably, both R¹ and R² are —H.

Preferably, X is —O—, —S—, —C(R³R⁴)—, —CH₂CH₂—, —CH═CH—CH₂—, —CH₂—CH═CH—, —CH₂—(C═O)—, —(C═O)—CH₂—, —CH₂CH₂CH₂—, —CH═CH—, —N(R⁵)—(C═O)—, —(C═O)—N(R⁵)—, —O—CH₂—, —CH₂—O—, —S—CH₂—, —CH₂—S—, —(C═O)—, —N(R⁷)— or —(S═O)—, or more preferably, —O—, —S—, —CH₂CH₂—, —CH═CH—, —O—CH₂—, —CH₂—O—, —OCH₂O—, —S—CH₂— or —CH₂—S—. In another preferred embodiment, X is —CH₂CH₂—, —CH₂—(C═O)—, —(C═O)—CH₂—, or —CH═CH—. More preferably, X is —CH₂CH₂—, —O—CH₂—, —CH₂—O—, or —CH═CH—. Most preferably, X is —CH₂CH₂—.

In another preferred embodiment, Y is >N—CH₂—, >CH—CH₂— or >C═CH—, or more preferably, >CH—CH₂— or >C═CH—. In a more preferred embodiment, Y is >C═CH—.

Z is preferably a group represented by a structural formula selected from Z^{a}-Z^{p}:

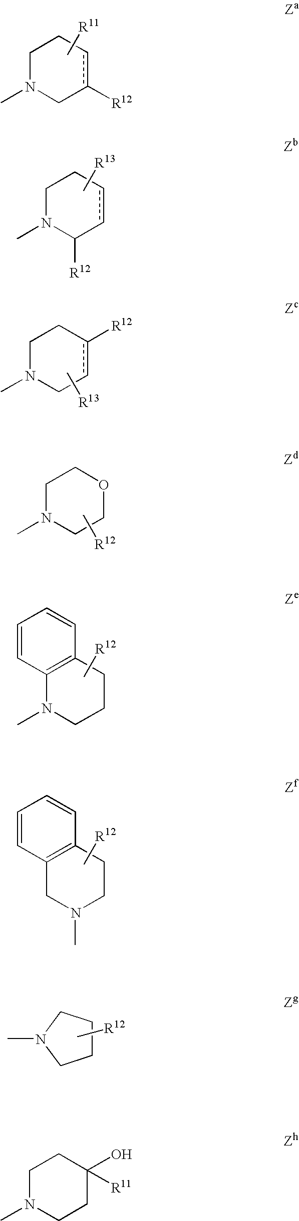

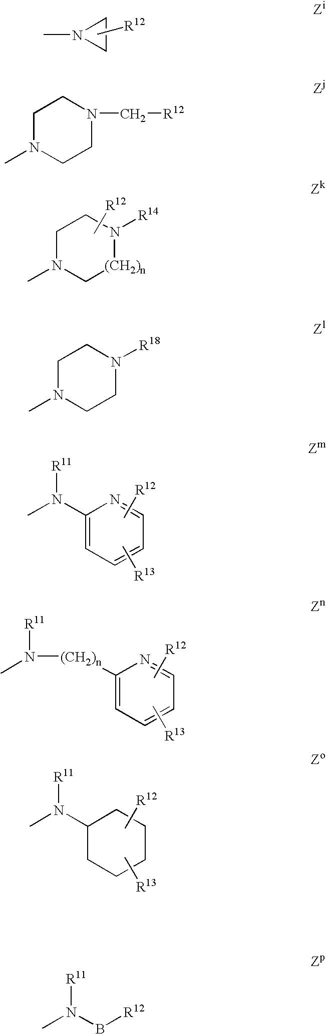

In structural formulas Z^{a}-Z^{p}, the variable n is 0, 1 or 2. R¹¹ and R¹² are as provided above. R¹³ is —H, halogen, trifluoromethyl, hydroxy, C₁₋₆-alkyl or C₁₋₆-alkoxy. R¹⁴ is —H or C₁₋₆-alkyl. The dashed/solid line pair “ [symbol] ”, e.g., in structural formulas Z^{a}-Z^{p}, is optionally a single bond or a double bond. R¹⁸ is selected from R^{18a-d}:

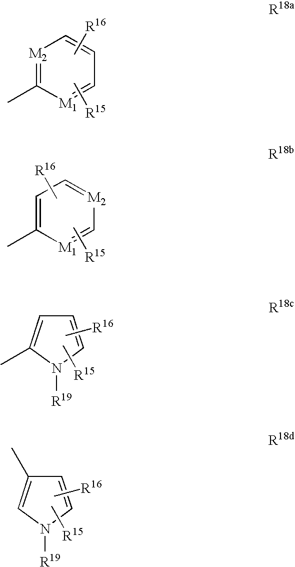

In structural formulas R^{18a-d}, M₁ and M₂ independently are C or N. R¹⁵ is —H, halogen, trifluoromethyl, nitro or cyano. R¹⁶ is —H, halogen, trifluoromethyl, nitro, cyano, —(CH₂)_{q}COR¹⁷, —(CH₂)_{q}OH or —(CH₂)_{q}SO₂R¹⁷, wherein q is 0, 1 or 2. Or, R¹⁶ is selected from structural formulas R^{16a-g}: 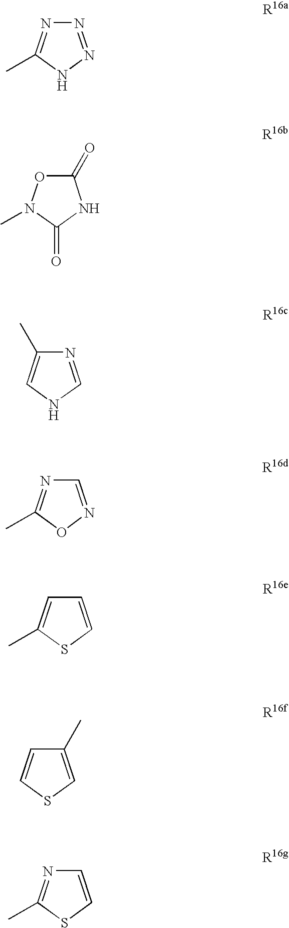
R¹⁹ is —H, C₁₋₆-alkyl, phenyl or benzyl.

Z is preferably selected from structural formulas Z^{a}-Z^{k}, wherein n is 1 or 2. Even more preferably, Z is selected from Z^{a}, Z^{b}, Z^{c}, Z^{g}, and Z^{l}.

In other preferred embodiments, the compound is represented by structural formulae I or I′, wherein the variables R¹, R², r, s, and A are as provided above, and the variables X, Y, and Z are selected from:

X is —O—, —S—, —O—CH₂—, —CH₂—O—, —CH₂—O—, —CH₂—O—CH₂—, —S—CH₂—, or —CH₂—S—; Y is >N—; and Z is a group represented by one of structural formulas Z^{a-d} or Z^{g-l}, wherein [symbol] is a double bond;

X is —O—, —S—, —O—CH₂—, —CH₂—O—, —O—CH₂—, —CH₂—O—CH₂—, —S—CH₂—, or —CH₂—S—; Y is >N—; and Z is a group represented by one of structural formulas Z^{a-d} or Z^{g-l}, wherein [symbol] is a single bond;

X is —O—, —S—, —O—CH₂—, —CH₂—O—, —O—CH₂—O—, —CH₂—O—CH₂—, —S—CH₂—, or —CH₂—S—; Y is >N—; and Z is a group represented by one of structural formulas Z^{e} or Z^{f};

X is —O—, —S—, —O—CH₂—, —CH₂—O—, —O—CH₂—O—, —CH₂—O—CH₂—, —S—CH₂—, or —CH₂—S—; Y is >N—; and Z is a group represented by one of structural formulas Z^{m} or Zⁿ;

X is —O—, —S—, —O—CH₂—, —CH₂—O—, —O—CH₂—O—, —CH₂—O—CH₂—, —S—CH₂—, or —CH₂—S—; Y is >N—; and Z is a group represented by one of structural formulas Z^{o} or Z^{p};

X is —O—, —S—, —O—CH₂—, —CH₂—O—, —O—CH₂—O—, —CH₂—O—CH₂—, —S—CH₂—, or —CH₂—S—; Y is >N—; and Z is a group represented by one of structural formulas Z^{j-l};

X is —O—, —S—, —O—CH₂—, —CH₂—O—, —O—CH₂—O—, —CH₂—O—CH₂—, —S—CH₂—, or —CH₂—S—; Y is >CH—; and Z is a group represented by one of structural formulas Z^{a-d} or Z^{g-l}, wherein [symbol] is a double bond;

X is —O—, —S—, —O—CH₂—, —CH₂—O—, —O—CH₂—O—, —CH₂—O—CH₂—, —S—CH₂—, or —CH₂—S—; Y is >CH—; and Z is a group represented by one of structural formulas Z^{a-d} or Z^{g-l}, wherein [symbol] is a single bond;

X is —O—, —S—, —O—CH₂—, —CH₂—O—, —O—CH₂—O—, —CH₂—O—CH₂—, —S—CH₂—, or —CH₂—S—; Y is >CH—; and Z is a group represented by one of structural formulas Z^{e} or Z^{f};

X is —O—, —S—, —O—CH₂—, —CH₂—O—, —O—CH₂—O—, —CH₂—O—CH₂—, —S—CH₂—, or —CH₂—S—; Y is >CH—; and Z is a group represented by one of structural formulas Z^{m} or Zⁿ;

X is —O—, —S—, —O—CH₂—, —CH₂—O—, —O—CH₂—O—, —CH₂—O—CH₂—, —S—CH₂—, or —CH₂—S—; Y is >CH—; and Z is a group represented by one of structural formulas Z^{o} or Z^{p};

X is —O—, —S—, —O—CH₂—, —CH₂—O—, —O—CH₂—O—, —CH₂—O—CH₂—, —S—CH₂—, or —CH₂—S—; Y is >CH—; and Z is a group represented by one of structural formulas Z^{j-l};

X is —O—, —S—, —O—CH₂—, —CH₂—O—, —O—CH₂—O—, —CH₂—O—CH₂—, —S—CH₂—, or —CH₂—S—; Y is >N—(C═O)—; and Z is a group represented by one of structural formulas Z^{a-d} or Z^{g-l}, wherein [symbol] is a double bond;

X is —O—, —S—, —O—CH₂—, —CH₂—O—, —CH₂—O—, —CH₂—O—CH₂—, —S—CH₂—, or —CH₂—S—; Y is >N—(C═O)—; and Z is a group represented by one of structural formulas Z^{a-d} or Z^{g-l} wherein [symbol] is a single bond;

X is —O—, —S—, —O—CH₂—, —CH₂—O—, —CH₂—O—, —CH₂—O—CH₂—, —S—CH₂—, or —CH₂—S—; Y is >N—(C═O)—; and Z is a group represented by one of structural formulas Z^{e} or Z^{f};

X is —O—, —S—, —O—CH₂—, —CH₂—O—, —O—CH₂—O—, —CH₂—O—CH₂—, —S—CH₂—, or —CH₂—S—; Y is >N—(C═O)—; and Z is a group represented by one of structural formulas Z^{m} or Zⁿ;

X is —O—, —S—, —O—CH₂—, —CH₂—O—, —O—CH₂—O—, —CH₂—O—CH₂—, —S—CH₂—, or —CH₂—S—; Y is >N—(C═O)—; and Z is a group represented by one of structural formulas Z^{o} or Z^{p};

X is —O—, —S—, —O—CH₂—, —CH₂—O—, —O—CH₂—O—, —CH₂—O—CH₂—, —S—CH₂—, or —CH₂—S—; Y is >N—(C═O)—; and Z is a group represented by one of structural formulas Z^{j-l};

X is —O—, —S—, —O—CH₂—, —CH₂O—, —O—CH₂—O—, —CH₂—O—CH₂—, —S—CH₂—, or —CH₂—S—; Y is >C═C(R⁸)—; and Z is a group represented by one of structural formulas Z^{a-d} or Z^{g-l}, wherein [symbol] is a double bond;

X is —O—, —S—, —O—CH₂—, —CH₂—O—, —CH₂—O—, —CH₂—O—CH₂—, —S—CH₂—, or —CH₂—S—; Y is >C═C(R⁸)—; and Z is a group represented by one of structural formulas Z^{a-d} or Z^{g-l}, wherein [symbol] is a single bond;

X is —O—, —S—, —O—CH₂—, —CH₂—O—, —O—CH₂—O—, —CH₂—O—CH₂—, —S—CH₂—, or —CH₂—S—; Y is >C═C(R⁸)—; and Z is a group represented by one of structural formulas Z^{e} or Z^{f};

X is —O—, —S—, —O—CH₂—, —CH₂—O—, —O—CH₂—O—, —CH₂—O—CH₂—, —S—CH₂—, or —CH₂—S—; Y is >C═C(R⁸)—; and Z is a group represented by one of structural formulas Z^{m} or Zⁿ;

X is —O—, —S—, —O—CH₂—, —CH₂—O—, —O—CH₂—O—, —CH₂—O—CH₂—, —S—CH₂—, or —CH₂—S—; Y is >C═C(R⁸)—; and Z is a group represented by one of structural formulas Z^{o} or Z^{p};

X is —O—, —S—, —O—CH₂—, —CH₂—O—, —O—CH₂—O—, —CH₂—O—CH₂—, —S—CH₂—, or —CH₂—S—; Y is >C═C(R⁸)—; and Z is a group represented by one of structural formulas Z^{j-l};

X is —CH₂CH₂CH₂—, —CH₂CH₂—, —C(R³R⁴)—, —CH(R⁶)CH₂—, or —CH₂CH(R⁶)—; Y is >N—; and Z is a group represented by one of structural formulas Z^{a-d} or Z^{g-l}, wherein [symbol] is a double bond;

X is —CH₂CH₂CH₂—, —CH₂CH₂—, —C(R³R⁴)—, —CH(R⁶)CH₂—, or —CH₂CH(R⁶)—; Y is >N—; and Z is a group represented by one of structural formulas Z^{a-d} or Z^{g-l}, wherein [symbol] is a single bond;

X is —CH₂CH₂CH₂—, —CH₂CH₂—, —C(R³R⁴)—, —CH(R)CH₂—, or —CH₂CH(R⁶)—; Y is >N—; and Z is a group represented by one of structural formulas Z^{e} or Z^{f};

X is —CH₂CH₂CH₂—, —CH₂CH₂—, —C(R³R⁴)—, —CH(R⁶)CH₂—, or —CH₂CH(R⁶)—; Y is >N—; and Z is a group represented by one of structural formulas Z^{m} or Zⁿ;

X is —CH₂CH₂CH₂—, —CH₂CH₂—, —C(R³R⁴)—, —CH(R⁶)CH₂—, or —CH₂CH(R⁶)—; Y is >N—; and Z is a group represented by one of structural formulas Z^{o} or Z^{p};

X is —CH₂CH₂CH₂—, —CH₂CH₂—, —C(R³R⁴)—, —CH(R⁶)CH₂—, or —CH₂CH(R⁶)—; Y is >N—; and Z is a group represented by one of structural formulas Z^{j-l};

X is —CH₂CH₂CH₂—, —CH₂CH₂—, —C(R³R⁴)—, —CH(R⁶)CH₂—, or —CH₂CH(R⁶)—; Y is >CH—; and Z is a group represented by one of structural formulas Z^{a-d} or Z^{g-l}, wherein [symbol] is a double bond;

X is —CH₂CH₂CH₂—, —CH₂CH₂—, —C(R³R⁴)—, —CH(R⁶)CH₂—, or —CH₂CH(R⁶)—; Y is >CH—; and Z is a group represented by one of structural formulas Z^{a-d} or Z^{g-l}, wherein [symbol] is a single bond;

X is —CH₂CH₂CH₂—, —CH₂CH₂—, —C(R³R⁴)—, —CH(R⁶)CH₂—, or —CH₂CH(R⁶)—; Y is >CH—; and Z is a group represented by one of structural formulas Z^{e} or Z^{f};

X is —CH₂CH₂CH₂—, —CH₂CH₂—, —C(R³R⁴)—, —CH(R⁶)CH₂—, or —CH₂CH(R⁶)—; Y is >CH—; and Z is a group represented by one of structural formulas Z^{m} or Zⁿ;

X is —CH₂CH₂CH₂—, —CH₂CH₂—, —C(R³R⁴)—, —CH(R⁶)CH₂—, or —CH₂CH(R⁶)—; Y is >CH—; and Z is a group represented by one of structural formulas Z^{o} or Z^{p};

X is —CH₂CH₂CH₂—, —CH₂CH₂—, —C(R³R⁴)—, —CH(R⁶)CH₂—, or —CH₂CH(R⁶)—; Y is >CH—; and Z is a group represented by one of structural formulas Z^{j-l};

X is —CH₂CH₂CH₂—, —CH₂CH₂—, —C(R³R⁴)—, —CH(R⁶)CH₂—, or —CH₂CH(R⁶)—; Y is >N—(C═O)—; and Z is a group represented by one of structural formulas Z^{a-d} or Z^{g-l}, wherein [symbol] is a double bond;

X is —CH₂CH₂CH₂—, —CH₂CH₂—, —C(R³R⁴)—, —CH(R⁶)CH₂—, or —CH₂CH(R)—; Y is >N—(C═O)—; and Z is a group represented by one of structural formulas Z^{a-d} or Z^{g-l}, wherein [symbol] is a single bond;

X is —CH₂CH₂CH₂—, —CH₂CH₂—, —C(R³R⁴)—, —CH(R⁶)CH₂—, or —CH₂CH(R⁶)—; Y is >N—(C═O)—; and Z is a group represented by one of structural formulas Z^{e} or Z^{f};

X is —CH₂CH₂CH₂—, —CH₂CH₂—, —C(R³R⁴)—, —CH(R⁶)CH₂—, or —CH₂CH(R⁶)—; Y is >N—(C═O)—; and Z is a group represented by one of structural formulas Z^{m} or Zⁿ;

X is —CH₂CH₂CH₂—, —CH₂CH₂—, —C(R³R⁴)—, —CH(R⁶)CH₂—, or —CH₂CH(R⁶)—; Y is >N—(C═O)—; and Z is a group represented by one of structural formulas Z^{o} or Z^{p} ;

X is —CH₂CH₂CH₂—, —CH₂CH₂—, —C(R³R⁴)—, —CH(R⁶)CH₂—, or —CH₂CH(R⁶)—; Y is >N—(C═O)—; and Z is a group represented by one of structural formulas Z^{j-l};

X is —CH₂CH₂CH₂—, —CH₂CH₂—, —C(R³R⁴)—, —CH(R⁶)CH₂—, or —CH₂CH(R⁶)—; Y is >C═C(R⁸)—; and Z is a group represented by one of structural formulas Z^{a-d} or Z^{g-l}, wherein [symbol] is a double bond;

X is —CH₂CH₂CH₂—, —CH₂CH₂—, —C(R³R⁴)—, —CH(R⁶)CH₂—, or —CH₂CH(R⁶)—; Y is >C═C(R⁸)—; and Z is a group represented by one of structural formulas Z^{a-d} or Z^{g-l}, wherein [symbol] is a single bond;

X is —CH₂CH₂CH₂—, —CH₂CH₂—, —C(R³R⁴)—, —CH(R⁶)CH₂—, or —CH₂CH(R⁶)—; Y is >C═C(R⁸)—; and Z is a group represented by one of structural formulas Z^{e} or Z^{f};

X is —CH₂CH₂CH₂—, —CH₂CH₂—, —C(R³R⁴)—, —CH(R⁶)CH₂—, or —CH₂CH(R⁶)—; Y is >C═C(R⁸)—; and Z is a group represented by one of structural formulas Z^{m} or Zⁿ;

X is —CH₂CH₂CH₂—, —CH₂CH₂—, —C(R³R⁴)—, —CH(R⁶)CH₂—, or —CH₂CH(R⁶)—; Y is >C═C(R⁸)—; and Z is a group represented by one of structural formulas Z^{o} or Z^{p};

X is —CH₂CH₂CH₂—, —CH₂CH₂—, —C(R³R⁴)—, —CH(R⁶)CH₂—, or —CH₂CH(R⁶)—; Y is >C═C(R⁸)—; and Z is a group represented by one of structural formulas Z^{j-l};

X is —CH═CH—CH₂—, —CH₂—CH═CH—, —CH═CH—, or ortho-phenylene; Y is >N—; and Z is a group represented by one of structural formulas Z^{a-d} or Z^{g-l}, wherein [symbol] is a double bond;

X is —CH═CH—CH₂—, —CH₂—CH═CH—, —CH═CH—, or ortho-phenylene; Y is >N—; and Z is a group represented by one of structural formulas Z^{a-d} or Z^{g-l}, wherein [symbol] is a single bond;

X is —CH═CH—CH₂—, —CH₂—CH═CH—, —CH═CH—, or ortho-phenylene; Y is >N—; and Z is a group represented by one of structural formulas Z^{e} or Z^{f};

X is —CH═CH—CH₂—, —CH₂—CH═CH—, —CH═CH—, or ortho-phenylene; Y is >N—; and Z is a group represented by one of structural formulas Z^{m} or Zⁿ;

X is —CH═CH—CH₂—, —CH₂—CH═CH—, —CH═CH—, or ortho-phenylene; Y is >N—; and Z is a group represented by one of structural formulas Z^{o} or Z^{p};

X is —CH═CH—CH₂—, —CH₂—CH═CH—, —CH═CH—, or ortho-phenylene; Y is >N—; and Z is a group represented by one of structural formulas Z^{j-l};

X is —CH═CH—CH₂—, —CH₂—CH═CH—, —CH═CH—, or ortho-phenylene; Y is >CH—; and Z is a group represented by one of structural formulas Z^{a-d} or Z^{g-l}, wherein [symbol] is a double bond;

X is —CH═CH—CH₂—, —CH₂—CH═CH—, —CH═CH—, or ortho-phenylene; Y is >CH—; and Z is a group represented by one of structural formulas Z^{a-d} or Z^{g-l}, wherein [symbol] is a single bond;

X is —CH═CH—CH₂—, —CH₂—CH═CH—, —CH═CH—, or ortho-phenylene; Y is >CH—; and Z is a group represented by one of structural formulas Z^{e} or Z^{f};

X is —CH═CH—CH₂—, —CH₂—CH═CH—, —CH═CH—, or ortho-phenylene; Y is >CH—; and Z is a group represented by one of structural formulas Z^{m} or Zⁿ;

X is —CH═CH—CH₂—, —CH₂—CH═CH—, —CH═CH—, or ortho-phenylene; Y is >CH—; and Z is a group represented by one of structural formulas Z^{o} or Z^{p};

X is —CH═CH—CH₂—, —CH₂—CH═CH—, —CH═CH—, or ortho-phenylene; Y is >CH—; and Z is a group represented by one of structural formulas Z^{j-l};

X is —CH═CH—CH₂—, —CH₂—CH═CH—, —CH═CH—, or ortho-phenylene; Y is >N—(C═O)—; and Z is a group represented by one of structural formulas Z^{a-d} or Z^{g-l}, wherein [symbol] is a double bond;

X is —CH═CH—CH₂—, —CH₂—CH═CH—, —CH═CH—, or ortho-phenylene; Y is >N—(C═O)—; and Z is a group represented by one of structural formulas Z^{a-d} or Z^{g-l}, wherein [symbol] is a single bond;

X is —CH═CH—CH₂—, —CH₂—CH═CH—, —CH═CH—, or ortho-phenylene; Y is >N—(C═O)—; and Z is a group represented by one of structural formulas Z^{e} or Z^{f};

X is —CH═CH—CH₂—, —CH₂—CH═CH—, —CH═CH—, or ortho-phenylene; Y is >N—(C═O)—; and Z is a group represented by one of structural formulas Z^{m} or Zⁿ;

X is —CH═CH—CH₂—, —CH₂—CH═CH—, —CH═CH—, or ortho-phenylene; Y is >N—(C═O)—; and Z is a group represented by one of structural formulas Z^{o} or Z^{p};

X is —CH═CH—CH₂—, —CH₂—CH═CH—, —CH═CH—, or ortho-phenylene; Y is >N—(C═O)—; and Z is a group represented by one of structural formulas Z^{j-l};

X is —CH═CH—CH₂—, —CH₂—CH═CH—, —CH═CH—, or ortho-phenylene; Y is >C═C(R⁸)—; and Z is a group represented by one of structural formulas Z^{a-d} or Z^{g-l}, wherein [symbol] is a double bond;

X is —CH═CH—CH₂—, —CH₂—CH═CH—, —CH═CH—, or ortho-phenylene; Y is >C═C(R⁸)—; and Z is a group represented by one of structural formulas Z^{a-d} or Z^{g-l}, wherein [symbol] is a single bond;

X is —CH═CH—CH₂—, —CH₂—CH═CH—, —CH═CH—, or ortho-phenylene; Y is >C═C(R⁸)—; and Z is a group represented by one of structural formulas Z^{e} or Z^{f};

X is —CH═CH—CH₂—, —CH₂—CH═CH—, —CH═CH—, or ortho-phenylene; Y is >C═C(R⁸)—; and Z is a group represented by one of structural formulas Z^{m} or Zⁿ;

X is —CH═CH—CH₂—, —CH₂—CH═CH—, —CH═CH—, or ortho-phenylene; Y is >C═C(R⁸)—; and Z is a group represented by one of structural formulas Z^{o} or Z^{p};

X is —CH═CH—CH₂—, —CH₂—CH═CH—, —CH═CH—, or ortho-phenylene; Y is >C═C(R⁸)—; and Z is a group represented by one of structural formulas Z^{j-l};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—; and Z is a group represented by one of structural formulas Z^{a-d} or Z^{g-l}, wherein [symbol] is a double bond;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—; and Z is a group represented by one of structural formulas Z^{a-d} or Z^{g-l}, wherein [symbol] is a single bond;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—; and Z is a group represented by one of structural formulas Z^{e} or Z^{f};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—; and Z is a group represented by one of structural formulas Z^{m} or Zⁿ;

X is —C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—; and Z is a group represented by one of structural formulas Z^{o} or Z^{p};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—; and Z is a group represented by one of structural formulas Z^{j-l};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >CH—; and Z is a group represented by one of structural formulas Z^{a-d} or Z^{g-l}, wherein [symbol] is a double bond;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >CH—; and Z is a group represented by one of structural formulas Z^{a-d} or Z^{g-l}, wherein [symbol] is a single bond;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >CH—; and Z is a group represented by one of structural formulas Z^{e} or Z^{f};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >CH—; and Z is a group represented by one of structural formulas Z^{m} or Zⁿ;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >CH—; and Z is a group represented by one of structural formulas Z^{o} or Z^{p};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >CH—; and Z is a group represented by one of structural formulas Z^{j-l};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—(C═O)—; and Z is a group represented by one of structural formulas Z^{a-d} or Z^{g-l}, wherein [symbol] is a double bond;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—(C═O)—; and Z is a group represented by one of structural formulas Z^{a-d} or Z^{g-l}, wherein [symbol] is a single bond;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—(C═O)—; and Z is a group represented by one of structural formulas Z^{e} or Z^{f};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—(C═O)—; and Z is a group represented by one of structural formulas Z^{m} or Zⁿ;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—(C═O)—; and Z is a group represented by one of structural formulas Z^{o} or Z^{p};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >N—(C═O)—; and Z is a group represented by one of structural formulas Z^{j-l};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >C═C(R⁸)—; and Z is a group represented by one of structural formulas Z^{a-d} or Z^{g-l}, wherein [symbol] is a double bond;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >C═C(R⁸)—; and Z is a group represented by one of structural formulas Z^{a-d} or Z^{g-l}, wherein [symbol] is a single bond;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >C═C(R⁸)—; and Z is a group represented by one of structural formulas Z^{e} or Z^{f};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >C═C(R⁸)—; and Z is a group represented by one of structural formulas Z^{m} or Zⁿ;

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >C═C(R⁸)—; and Z is a group represented by one of structural formulas Z^{o} or Z^{p};

X is —(C═O)—, —(S═O)—, —CH₂—(C═O)—, —(C═O)—CH₂—, —N(R⁵)—(C═O)—, —(C═O)—N(R⁵)—, —N(CH₃)SO₂—, or —SO₂N(CH₃)—; Y is >C═C(R⁸)—; and Z is a group represented by one of structural formulas Z^{j-l};

X is —(CH₂)N(R⁵)—, —N(R⁵)(CH₂)—, or —N(R⁷)—; Y is >N—; and Z is a group represented by one of structural formulas Z^{a-d} or Z^{g-l}, wherein [symbol] is a double bond;

X is —(CH₂)N(R⁵)—, —N(R⁵)(CH₂)—, or —N(R⁷)—; Y is >N—; and Z is a group represented by one of structural formulas Z^{a-d} or Z^{g-l}, wherein [symbol] is a single bond;

X is —(CH₂)N(R⁵)—, —N(R⁵)(CH₂)—, or —N(R⁷)—; Y is >N—; and Z is a group represented by one of structural formulas Z^{e} or Z^{f};

X is —(CH₂)N(R⁵)—, —N(R⁵)(CH₂)—, or —N(R⁷)—; Y is >N—; and Z is a group represented by one of structural formulas Z^{m} or Zⁿ;

X is —(CH₂)N(R⁵)—, —N(R⁵)(CH₂)—, or —N(R⁷)—; Y is >N—; and Z is a group represented by one of structural formulas Z^{o} or Z^{p};

X is —(CH₂)N(R⁵)—, —N(R⁵)(CH₂)—, or —N(R⁷)—; Y is >N—; and Z is a group represented by one of structural formulas Z^{j-l};

X is —(CH₂)N(R⁵)—, —N(R⁵)(CH₂)—, or —N(R⁷)—; Y is >CH—; and Z is a group represented by one of structural formulas Z^{a-d} or Z^{g-l}, wherein [symbol] is a double bond;

X is —(CH₂)N(R⁵)—, —N(R⁵)(CH₂)—, or —N(R⁷)—; Y is >CH—; and Z is a group represented by one of structural formulas Z^{a-d} or Z^{j-l}, wherein [symbol] is a single bond;

X is —(CH₂)N(R⁵)—, —N(R⁵)(CH₂)—, or —N(R⁷)—; Y is >CH—; and Z is a group represented by one of structural formulas Z^{e} or Z^{f};

X is —(CH₂)N(R⁵)—, —N(R⁵)(CH₂)—, or —N(R⁷)—; Y is >CH—; and Z is a group represented by one of structural formulas Z^{m} or Zⁿ;

X is —(CH₂)N(R⁵)—, —N(R⁵)(CH₂)—, or —N(R⁷)—; Y is >CH—; and Z is a group represented by one of structural formulas Z^{o} or Z^{p};

X is —(CH₂)N(R⁵)—, —N(R⁵)(CH₂)—, or —N(R⁷)—; Y is >CH—; and Z is a group represented by one of structural formulas Z^{j-l};

X is —(CH₂)N(R⁵)—, —N(R⁵)(CH₂)—, or —N(R⁷)—; Y is >N—(C═O)—; and Z is a group represented by one of structural formulas Z^{a-d} or Z^{g-l}, wherein [symbol] is a double bond;

X is —(CH₂)N(R⁵)—, —N(R⁵)(CH₂)—, or —N(R⁷)—; Y is >N—(C═O)—; and Z is a group represented by one of structural formulas Z^{a-d} or Z^{g-l}, wherein [symbol] is a single bond;

X is —(CH₂)N(R⁵)—, —N(R⁵)(CH₂)—, or —N(R⁷)—; Y is >N—(C═O)—; and Z is a group represented by one of structural formulas Z^{e} or Z^{f};

X is —(CH₂)N(R⁵)—, —N(R⁵)(CH₂)—, or —N(R⁷)—; Y is >N—(C═O)—; and Z is a group represented by one of structural formulas Z^{m} or Zⁿ;

X is —(CH₂)N(R⁵)—, —N(R⁵)(CH₂)—, or —N(R⁷)—; Y is >N—(C═O)—; and Z is a group represented by one of structural formulas Z^{o} or Z^{p};

X is —(CH₂)N(R⁵)—, —N(R⁵)(CH₂)—, or —N(R⁷)—; Y is >N—(C═O)—; and Z is a group represented by one of structural formulas Z^{j-l};

X is —(CH₂)N(R⁵)—, —N(R⁵)(CH₂)—, or —N(R⁷)—; Y is >C═C(R⁸)-; and Z is a group represented by one of structural formulas Z^{a-d} or Z^{g-l}, wherein [symbol] is a double bond;

X is —(CH₂)N(R⁵)—, —N(R⁵)(CH₂)—, or —N(R⁷)—; Y is >C═C(R⁸)—; and Z is a group represented by one of structural formulas Z^{a-d} or Z^{g-l}, wherein [symbol] is a single bond;

X is —(CH₂)N(R⁵)—, —N(R⁵)(CH₂)—, or —N(R⁷)—; Y is >C═C(R⁸)—; and Z is a group represented by one of structural formulas Z^{e} or Z^{f};

X is —(CH₂)N(R⁵)—, —N(R⁵)(CH₂)—, or —N(R⁷)—; Y is >C═C(R⁸)—; and Z is a group represented by one of structural formulas Z^{m} or Zⁿ;

X is —(CH₂)N(R⁵)—, —N(R⁵)(CH₂)—, or —N(R⁷)—; Y is >C═C(R⁸)—; and Z is a group represented by one of structural formulas Z^{o} or Z^{p}; and

X is —(CH₂)N(R⁵)—, —N(R⁵)(CH₂)—, or —N(R⁷)—; Y is >C═C(R⁸)—; and Z is a group represented by one of structural formulas Z^{j-l}.

In other embodiments, the compound is represented by structural formula I, the variables R¹, R², r, s, X, Y, and Z are as provided above, and the variable A is —CH═CR⁹—, —CR⁹═CH—, —C≡C—, phenylene, C₃₋₇-cycloalkylene or the completion of a bond. In other preferred embodiments, A is —(C═O)— or —(C═CH₂)—; or A is —CH═CR⁹— or —CR⁹═CH—; or, A is —(CR⁹R¹⁰)— or —CH(OR²³)—. In still another embodiment, A is —CH(NHR³)—. R⁹, R¹⁰ and R²³ are as provided above. Most preferably, A is the completion of a bond.

In other embodiments, the compound is represented by structural formula I′. Preferably, r is 1, 2, 3, or 4; X is —O—, —S—, —O—CH₂—, —CH₂—O—, —CH₂—O—CH₂—, —CH₂CH₂CH₂—, —CH₂CH₂—, —C(R³R⁴)—, —CH(R⁶)CH₂—, —CH₂CH(R⁶)—, —CH═CH—, —(C═O)—, or —(S═O)—; Y is >N—, >CH—, or >C═CH—; and Z is Z^{a}-Z^{l}, wherein n is 1 or 2. More preferably, r is 1, 2, or 3; X is —O—CH₂—, —CH₂—O—, —CH₂—O—CH₂—, —CH₂CH₂CH₂—, —CH₂CH₂—, or —CH═CH—; Y is >CH— or >C═CH—; Z is Z is Z^{a}, Z^{b}, Z^{c}, or Z^{h}. Even more preferably, X is —CH₂—O—CH₂—, —CH₂CH₂CH₂—, or —CH₂CH₂—; Y is >C═CH—; and Z is Z^{a}, Z^{b}, or Z^{c}, wherein [symbol] is a single bond. In preferred embodiments, R¹ and R² independently are —H, halogen, hydroxy, —CN, —NO₂, optionally halogenated C₁₋₆-alkyl or C₁₋₆-alkoxy; or more preferably, R¹ and R² independently are —H, halogen, hydroxy, C₁₋₆-alkyl or C₁₋₆-alkoxy; or still more preferably, R¹ and R² independently are —H or halogen.

In other preferred embodiments, the compound is represented by structural formula II: 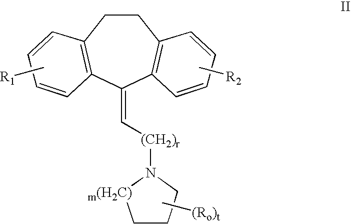
wherein

R¹ and R² independently are —H, halogen, hydroxy, —CN, —NO₂, optionally halogenated C₁₋₆-alkyl or C₁₋₆-alkoxy, —NR²¹R²², —SO₂)NR²¹R²², —NR²¹(SO₂)NR²², —(CO)N²¹R²², —NR²¹(CO)R²², —(CO)R²², or —(CO₂)R²², and R²¹ and R²² independently are —H or C₁₋₆alkyl;

Rₒ is optionally halogenated C₁₋₆-alkyl or C₁₋₆-alkoxy, or phenyl optionally substituted with halogen, or hydroxy, or nitro or cyano, or —(CH₂)_{q}COR^{p}, or —(CH₂)_{q}CONHSO₂Aryl, or —(CH₂)_{q}CONHSO₂Heteroaryl or —(CH₂)_{q}CONHS(O)₂-Alkyl or —(CH₂)_{q}OH or —(CH₂)_{q}SO₂R^{p}, or, —(CH₂)_{q}S(O)₂NHCO-alkyl, or —(CH₂)_{q}S(O)₂NHCO-aryl, or —(CH₂)_{q}S(O)NHCO-alkyl, or —(CH₂)_{q}S(O)NHCO-aryl, or —(CH₂)_{q}P(O)(OH)₂, or —(CH₂)_{q}P(O)OH, or —(CH₂)_{q}

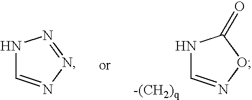

wherein q is 0, 1 or 2;

R^{p} is H, OH or C₁₋₈alkyl,

r is 1, 2, 3 or4;

m is 1 or 2, and

t is 1, 2, or 3, such that there are 1, 2, or 3 Rₒ substituents on the nitrogen containing ring.

In other preferred embodiments, the compound is represented by structural formula IIIa or IIIb:

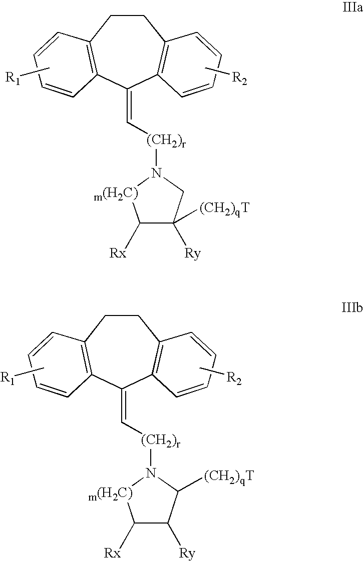

wherein

R¹ and R² independently are —H, halogen, hydroxy, —CN, —NO₂, optionally halogenated C₁₋₆-alkyl or C₁₋₆-alkoxy, —NR²¹R²², —(SO₂)NR²¹R¹², —NR²¹(SO₂)NR²², —(CO)NR²¹R²², —NR²¹(CO)R²², —(CO)R²², or —(CO₂)R²², and R²¹ and R²² independently are —H or C₁₋₆-alkyl;

Rₓ and R_{y} are, independently, hydrogen, optionally halogenated C₁₋₆-alkyl or C₁₋₆-alkoxy, or, taken together, Rₓ and R_{y} form a bond;

T is COOH, or COOR^{a} or CONHSO₂Aryl, or CONHSO₂Heteroaryl or CONHS(O)₂Alkyl or SO₃H, or, S(O)₂NHCOAlkyl, or S(O)₂NHCOAryl, or S(O)NHCOAlkyl, or S(O)NHCO-aryl, or P(O)(OH)₂, or

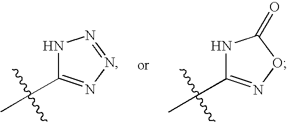

R¹ is C₁-C₆ alkyl;

q is 0, 1 or 2;

r is 1, 2, 3 or 4; and

m is 1 or 2.

In other preferred embodiments, the compound is represented by structural formula IVa or IVb:

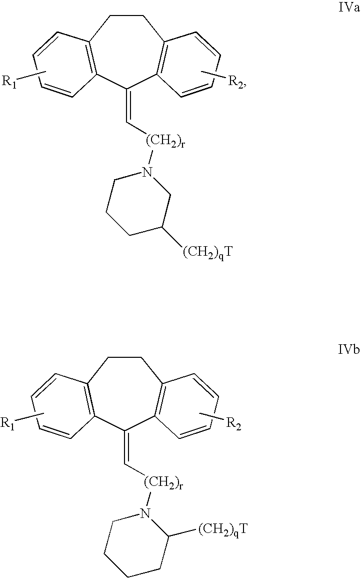

wherein

R¹ and R² independently are —H, halogen, hydroxy, —CN, —NO₂, optionally halogenated C₁₋₆-alkyl or C₁₋₆-alkoxy, —NR²¹R²², —(SO₂)NR²¹R²², —NR²¹(SO₂)NR²², —(CO)NR²¹R²², —NR²¹(CO)R²², —(CO)R²², or —(CO₂)R²², and R²¹ and R²² independently are —H or C₁₋₆-alkyl;

T is COOH, or COOR^{a} or CONHSO₂Aryl, or CONHSO₂Heteroaryl or CONHS(O)₂Alkyl or SO₃H, or, S(O)₂NHCOAlkyl, or S(O)₂NHCOAryl, or S(O)NHCOAlkyl, or S(O)NHCO-aryl, or P(O)(OH)₂, or

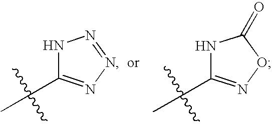

R^{a} is C₁-C₆ alkyl;

q is 0, 1 or 2 and

r is 1, 2, 3 or 4.

In other preferred embodiments, the compound is represented by structural formula Va or Vb:

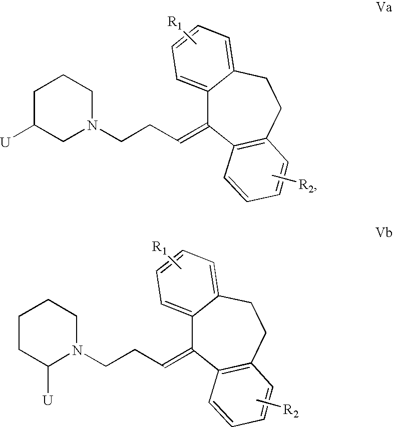

wherein

R¹ and R² independently are —H, halogen, hydroxy, —CN, —NO₂, optionally halogenated C₁₋₆-alkyl or C₁₋₆-alkoxy, —NR²¹R²², —(SO₂)NR²¹R²², —NR²¹(SO₂)NR²², (CO)NR²¹R²², —NR²¹(CO)R²², —(CO)R²², or —(CO₂)R²², R²¹ and R²² independently are —H or C₁₋₆-alkyl;

R^{a} is C₁-C₆ alkyl;

and U is COOH, or COOR^{a} or CONHSO₂Aryl, or

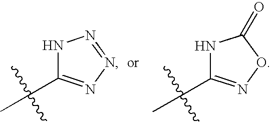

In preferred embodiments, R¹ and R² are hydrogen or halogen.

In preferred embodiments, the compound is selected from:

(R)-1-3-(10,11-Dihydro-5H-dibenzo[a,d]cyclohepten-5-ylidene)-1-propyl)-3-piperidinecarboxylic acid;

(S)-1-(3-(10,11-Dihydro-5H-dibenzo[a,d]cyclohepten-5-ylidene)-1-propyl)-3-piperidinecarboxylic acid;

(1-(3-(10,11-Dihydro-5H-dibenz[b,f]azepin-5-yl)-1-propyl)-3-piperidinyl)methanol;

(2S,4R)-1-(3-(10,11-Dihydro-5H-dibenz[b,f]azepin-5-yl)-1-propyl)-4-hydroxy-2-pyrrolidinecarboxylic acid;

(3-10,11-Dihydro-5H-dibenzo[b,f]azepin-5-yl)-2-hydroxy-propyl)-4-piperidinecarboxylic acid;

(4-(3-(10,11-Dihydro-5H-dibenz[b,f]azepin-5-yl)-1-propyl)piperazin-1-yl)acetic acid;

(R)-1-((2R)-Methyl-3-(3-methyl-10,11-dihydro-5H-dibenzo[b,f]azepin-5-yl)-1-propyl)-4-piperidinecarboxylic acid;

(R)-1-(10,11-Dihydro-5H-dibenzo[a,d]cyclohepten-5-yl)methyl)-3-piperidinecaxboxylic acid;

(R)-1-(2-(10,11-Dihydro-5H-dibenzo[a,d]cyclohepten-5-ylidene)-1-propyl)-3-piperidinecarboxylic acid;

(R)-1-(2-(10,11-dihydro-5H-dibenzo[b,f]azepin-5-yl)-1-methyl-ethyl)-3-piperidinecarboxylic acid;

(R)-1-(2-(10,11-Dihydro-5H-dibenzo[b,f]azepin-5-ylcarbonyl)-1-benzyl)-3-piperidinecarboxylic acid;

(R)-1-(2-(10,11-Dihydro-5H-dibenzo[b,f]azepin-5-ylmethyl)-1-pentyl)-3-piperidinecarboxylic acid;

(R)-1-(2-(10,11-Dihydro-5H-dibenzo[b,f]azepin-5-ylmethyl)-benzyl)-3-piperidinecarboxylic acid;

(R)-1-(2-(N-(10,11-dihydro-5H-dibenzo[a,d]cyclohepten-5-yl)-N-methylamino)ethyl)-3-piperidinecarboxylic acid

(R)-1-(3-(10,11-Dihydro-5H-dibenzo[a,d]cyclohepten-5-yl)-3-oxo-1-propyl)-3-piperidinecarboxylic acid;

(R)-1-(3-10,11-Dihydro-5H-benzo[a,d]cyclohepten-5-ylidene)-1-propyl)-2-piperidinecarboxylic acid;

(R)-1-(3-(10,11-Dihydro-5H-dibenzo[a,d]cyclohepten-5-ylidene)-1-propyl)-2-pyrrolidinecarboxylic acid;

(R)-1-(3-(10,11-Dihydro-5H-dibenzo[a,d]cyclohepten-5-ylidene)-1-propyl)-3-piperidinecarboxamide;

(R)-1-(3-(10,11-Dihydro-5H-dibenzo[b,f]azepin-5-yl)1-methylpropyl)-3-piperidinecarboxylic acid;

(R)-1-(3-(10,11-Dihydro-5H-dibenzo[b,f]azepin-5-yl)-1-propionyl)-3-peridinecarboxylic acid;

(R)-1-(3-(10,11-Dihydro-5H-dibenzo[b,f]azepin-5-yl)-2-hydroxypropyl)-3-peridinecarboxylic acid;

(R)-1-(3-(10,11-Dihydro-5H-dibenzo[b,f]azepin-5-yl)-3-oxopropyl)-3-peridinecarboxylic acid;

(R)-1-(4-(10,11--Dihydro-5H-dibenzo[a,d]cyclohepten-5-yl)-benzyl)-3-piperidinecarboxylic acid;

(R)-1-(4-(10,11-Dihydro-5H-dibenzo[b,f]azepin-5-yl)-2-butyn-1-yl)-3-piperidinecarboxylic acid;

(S)-1-(3-(10,11-Dihydro-5H-dibenzo[a,d]cyclohepten-5-ylidene)-1-propyl)-2-pyrrolidinecarboxylic acid;

1-(2-(10,11-Dihydro-5H-dibenzo[a,d]cyclohepten-5-yl)-1-ethyl)-(3R)-piperidinecarboxylic acid;

1-(2-(10,11-Dihydro-5H-dibenzo[a,d]cyclohepten-5-yl)-1-ethyl)-(3R)-piperidinecarboxylic acid;

1-(2-(10,11-Dihydro-5H-dibenzo[a,d]cyclohepten-5-ylidene)-1-propyl)-(3R)-piperidinecarboxylic acid;

1-(2-(10,11-Dihydro-5H-dibenzo[b,f]azepin-5-yl)-1-methyl-1-ethyl)-(3R)-piperidinecarboxylic acid;

1-(2-(10,11-Dihydro-5H-dibenzo[b,f]azepin-5-yl)-cyclopentylmethyl)-(3R)-piperidinecarboxylic acid;

1-(2-(10,11-Dihydro-5H-dibenzo[b,f]azepin-5-yl)-cyclopropyhnethyl)-(3R)-piperidinecarboxylic acid;

1-(2-(10,11-Dihydro-dibenzo[b,f]azepin-5-ylmethyl)-1-pentyl)-(3R)-piperidinecarboxylic acid;

1-(3-(10,11-Dihydro-5H-dibenz[b,f]azepin-5-yl)-1-propyl)-²-aziridinecarboxylic acid;

1-(3-(10,11-Dihydro-5H-dibenz[b,f]azepin-5-yl)-1-propyl)-2-methyl-3-peridinecarboxylic acid;

1-(3-(10,11-Dihydro-5H-dibenz[b,f]azepin-5-yl)-1-propyl)-2-pipendinecarboxylic acid;

1-(3-(10,11-Dihydro-5H-dibenz[b,f]azepin-5-yl)-1-propyl)-3-piperidineacetic acid;

1-(3-(10,11-Dihydro-5H-dibenz[b,f]azepin-5-yl)- -propyl)-3-piperidinecarboxamide;

1-(3-(10,11-Dihydro-5H-dibenz[b,f]azepin-5-yl)-1-propyl)-3-piperidinecarboxylic acid hydroxamide;

1-(3-(10,11-Dihydro-5H-dibenz[b,f]azepin-5-yl)-1-propyl)-3-quinuclidinium-carboxylate;

1-(3-(10,11-Dihydro-5H-dibenz[b,f]azepin-5-yl)-1-propyl)-4-methyl-[1,4]-diazepane-6-carboxylic acid;

1-(3-(10,11-Dihydro-5H-dibenz[b,f]azepin-5-yl)-1-propyl)-4-piperidineacetic acid;

1-(3-(10,11-Dihydro-5H-dibenz[b,f]azepin-5-yl)-1-propyl)-4-piperidinecarboxylic acid;

1-(3-(10,11-Dihydro-5H-dibenzo[a,d]cyclohepten-5-ylidene)-1-propionyl)-(3R)-piperidinecarboxylic acid;

1-(3-(10,11-Dihydro-5H-dibenzo[a,d]cyclohepten-5-ylidene)-1-propyl)-2-piperidinecarboxylic acid;

1-(3-(10,11-Dihydro-5H-dibenzo[a,d]cyclohepten-5-ylidene)-1-propyl)-4-piperidinecarboxylic acid;

1-(3-(10,11-Dihydro-5H-dibenzo[b,d]azepin-5-yl)-(2R)-methyl-1-propyl)-4-piperidinecarboxylic acid;

1-(3-(10,11-Dihydro-5H-dibenzo[b,f]azepin-5-yl)-(2R)-methyl-1-propyl)-(2R)-piperidinecarboxylic acid;

1-(3-(10,11-Dihydro-5H-dibenzo[b,f]azepin-5-yl)-(2R)-methyl-1-propyl)-(3R)-piperidinecarboxylic acid;

1-(3-(10,11-Dihydro-5H-dibenzo[b,f]azepin-5-yl)-(2R)-methyl-1-propyl)-3-pyrrolidinylacetic acid:

1-(3-(10,11-Dihydro-5H-dibenzo[b,f]azepin-5-yl)-(2R)-methyl-1-propyl)-4-piperidinecarboxylic acid;

1-(3-(10,11-Dihydro-5H-dibenzo[b,f]azepin-5-yl)-1-methyl-1-propyl)-(3R)-piperidinecarboxylic acid;

1-(3-(10,11-Dihydro-5H-dibenzo[b,f]azepin-5-yl)-1-propionyl)4-piperidinecarboxylic acid;

1-(3-(10,11-Dihydro-5H-dibenzo[b,f]azepin-5-yl)-2-hydroxy-1-propyl)-(3R)-piperidinecarboxylic acid;

1-(3-(10,11-Dihydro-5H-dibenzo[b,f]azepin-5-yl)-2-methyl-3-oxopropyl)-(3R)-piperidinecarboxylic acid;

1-(3-(10,11-Dihydro-5H-dibenzo[b,f]azepin-5-yl)-2-methyl-3-oxo-propyl)-3-piperidinearboxylic acid;

1-(3-(10,11-Dihydro-5H-dibenzo[b,f]azepin-5-yl)-2-propoxypropyl)-4-piperidinecarboxylic acid;

1-(3-(10OH-Phenoxazin-10-yl)-1-propyl)-4-piperidinecarboxylic acid;

1-(3-(2,8-Dibromo-10,11-dihydro-5H-dibenz[b,f]azepin-5-yl)-1-propyl)-4-piperidinecarboxylic acid;

1-(3-(2-Chloro-10,11-dihydro-5H -dibenzo[b,d]azepin-5-yl)-(2R)-methyl-1-propyl)-(3R)-piperidinecarboxylic acid;

1-(3-(3,7-Dichloro-10,11-dihydro-5H-dibenz[b,f]azepin-5-yl)-1-propyl)-4-piperidinecarboxylic acid;

1-(3-(3,7-Dimethyl-10,11-dihydro-5H-dibenz[b,f]azepin-5-yl)-1-propyl)-4-piperidinecarboxylic acid;

1-(3-(3-Chloro-10,11-dihydro-5H-dibenz[b,f]azepin-5-yl)-1-propyl)-4-piperidinecarboxylic acid;

1-(3-(3-Chloro-10,11-dihydro-5H-dibenzo[b,d]azepin-5-yl)-(2R)-methyl-1-propyl)-(3-piperidinecarboxylic acid;

1-(3-(3-Chloro-10,11-dihydro-5H-dibenzo[b,f]azepin-5-yl)-(2R)-methylpropyl)-4-piperidinecarboxylic acid;

1-(3-(3-Dimethylamino-10,11-dihydro-5H-dibenz[b,f]azepin-5-yl)-1-propyl)-4-piperidinecarboxylic acid;

1-(3-(3-Methoxy-10,11-dihydro-5H-dibenzo[b,f]azepin-5-yl)-(2R)-methyl-1-propyl)-(3R)-piperidinecarboxylic acid;

1-(3-(3-Methyl-10,11-dihydro-5H-dibenzo[a,d]cyclohepten-5-ylidene)-1-propyl-4-piperidinecarboxylic acid;

1-(3-(3-Methyl-10,11-dihydro-5H-dibenzo[a,d]cyclohepten-5-ylidene)-4-piperidinecarboxylic acid;

1-(3-(3-Methyl-10,11-dihydro-5H-dibenzo[b,f]azepin-5-yl)-(2R)-methyl-1-propyl)-(3R)-piperidinecarboxylic acid;

1-(3-(3-Trifluoromethyl-10,11-dihydro-5H-dibenzo[b,f]azepin-5-yl)-(2R)-methyl-1-propyl)-(3R)-piperidinecarboxylic acid;

1-(4-(10,11-Dihydro-5H-dibenzo[b,f]azepin-5-yl)-(2E)-butenyl)-(3R)-piperidinecarboxylic acid;

1-(4-(10,11-Dihydro-5H-dibenzo[b,f]azepin-5-yl)-(2Z)-butenyl)-(3R)-piperidinecarboxylic acid;

1-(4-(10,11-Dihydro-5H-dibenzo[b,f]azepin-5-yl)-2-butynyl)-(3R)-piperidinecarboxylic acid;

2-(1-(3-(10,11-Dihydrodibenzo[b,f]azepin-5-yl)-(2R)-methylpropyl)-4-piperazinyl)-nicotinic acid;

2-(3-(10,11-Dihydro-5H-dibenz[b,f]azepin-5-yl)-1-propyl)-1,2,3,4-tetrahydro-3-isoquinolinecarboxylic acid;

2-(3-(10,11-Dihydro-5H-dibenz[b,f]azepin-5-yl)-1-propyl)-1,2,3,4-tetrahydro-4-isoquinolinecarboxylic acid;

2-(4-(3-(10,11-Dihydro-5H-dibenzo[b,f]azepin-5-yl)-(2R)-methyl-1-propyl)-1-piperazinyl)-nicotinic acid;

2-(4-(3-(10,11-Dihydro-5H-dibenzo[b,f]azepin-5-yl)-2-hydroxypropyl)piperazin-1-yl)nicotinic acid;

4-(3-(10,11-Dihydro-5H-dibenz[b,f]azepin-5-yl)-1-propyl)-2-morpholinecarboxylic acid;

4-(3-(10,11-Dihydro-5H-dibenz[b,f]azepin-5-yl)-1-propyl)-2-piperazinecarboxylic acid; and

4-(4-Chlorophenyl)-1-(3-(10,11-dihydro-5H-dibenz[b,f]azepin-5-yl)-1-propyl)-4-piperidinol.

Where a compound is recited as an enantiomer, it is understood that the optical isomer is also included in the methods of the invention.

In a preferred embodiment, the compound is compound 1:

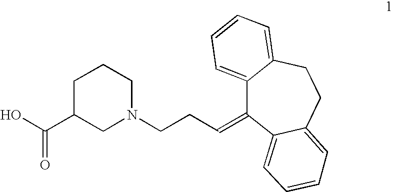

In another preferred embodiment, the compound is the R isomer of 1, represented by structural formula 1-R.

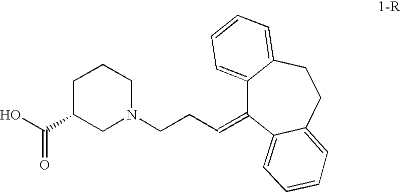

Still another preferred embodiment of the compound is the S isomer of 1, represented by structural formula 1-S.

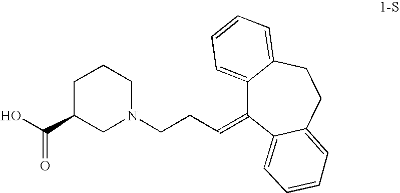

In another embodiment, the invention involves modulating sleep by administering a therapeutically effective amount of a compound of the invention, or a pharmaceutically effective salt thereof, to a subject. The method modulates sleep several ways including decreasing the time to sleep onset, increasing the average sleep bout length, and increasing the maximum sleep bout length.

A “subject” includes mammals, e.g., humans, companion animals (e.g. dogs, cats, birds, and the like), farm animals (e.g., cows, sheep, pigs, horses, fowl, and the like) and laboratory animals (e.g., rats, mice, guinea pigs, birds, and the like). Most preferably, the subject is human.

A subject in need of treatment has a sleep disorder that can affect the subject's ability to fall asleep and/or remain asleep, and/or results in unrefreshing sleep.

A subject in need of treatment has a sleep disorder which affects the subject's ability to fall asleep and/or remain asleep, and/or results in unrefreshing sleep. The term “sleep disorder” includes insomnia, night terrors, bruxism, somnambulism, sleep apnea, restless leg syndrome, unrefreshing sleep, seasonal affective disorder, circadian rhythm adjustment disorders, and the like. Insomnia is typically classed into sleep onset insomnia, where a subject takes more than 30 minutes to fall asleep; and sleep maintenance insomnia, where the subject spends more than 30 minutes awake during an expected sleep period, or, for example, waking before the desired wake-up time with an inability to get back to sleep. “Sleep disorders” include both endogenous disorders, such as sleep apnea, and disorders related to behavioral or external environmental factors. For example, “sleep disorders” include a subject's difficulty in adjusting to a new circadian rhythm, for example, due to “jet lag”; night, extended, or irregular work shifts; and the like. A “sleep disorder” can also arise in a subject that has other disorders, diseases, or injuries, or in a subject being treated with other medications, where the subject as a result has difficulty falling asleep and/or remaining asleep, or experiences unrefreshing sleep. For example, the disclosed method is useful for inducing sleep in a subject having difficulty sleeping as the result of undergoing chemotherapy, or as a result of injuries, or as the result of stress or mood disorders such as depression, anxiety, and the like.

As used herein, the term “sleep disorder” includes conditions recognized by one skilled in the art as sleep disorders, for example, conditions known in the art or conditions which are proposed to be sleep disorders or discovered to be sleep disorders. See, for example, Thorpy, M J International Classification of Sleep Disorders, Revised: Diagnostic and Coding Manual. American Sleep Disorders Association; Rochester, Minn. 1997; and ICD-9-CM, International Classification of Diseases, Ninth Revision, Clinical Modification, National Center for Health Statistics, Hyattsville, Md.

For example, sleep disorders can be generally classed into dyssomnias, e.g. intrinsic, extrinsic, and circadian rhythm disorders; parasomnias, e.g., arousal, sleep-wake transition, and rapid eye movement (REM) associated disorders, and other parasomnias; disorders associated with mental, neurological, and other medical disorders; and other sleep disorders.

Intrinsic sleep disorders include, for example, psychophysiological insomnia, sleep state misperception, idiopathic insomnia, narcolepsy, recurrent hypersomnia, idiopathic hypersomnia, post-traumatic hypersomnia, obstructive sleep apnea syndrome, central sleep apnea syndrome, central alveolar hypoventilation syndrome, periodic limb movement disorder, restless legs syndrome, and the like.

Extrinsic sleep disorders include, for example, inadequate sleep hygiene, environmental sleep disorder, altitude insomnia, adjustment sleep disorder, insufficient sleep syndrome, limit-setting sleep disorder, sleep-onset association disorder, food allergy insomnia, nocturnal eating (drinking) syndrome, hypnotic-dependent sleep disorder, stimulant-dependent sleep disorder, alcohol-dependent sleep disorder, toxin-induced sleep disorder, and the like.

Circadian rhythm sleep disorders include, for example, time-zone change Get lag) syndrome, shift work sleep disorder, irregular sleep-wake pattern, delayed sleep phase syndrome, advanced sleep phase syndrome, non-24-h sleep-wake disorder, and the like.

Arousal sleep disorders include, for example, confusional arousals, sleepwalking, sleep terrors, and the like.

Sleep-wake transition disorders include, for example, rhythmic movement disorder, sleep starts, sleeptalking, nocturnal leg cramps, and the like.

REM-associated sleep disorders include, for example, nightmares, sleep paralysis, impaired sleep-related penile erections, sleep-related painful erections, REM sleep-related sinus arrest, REM sleep behavior disorders, and the like.

Other parasomnias include, for example, sleep bruxism, sleep enuresis, sleep-related abnormal swallowing syndrome, nocturnal paroxysmal dystonia, sudden unexplained nocturnal death syndrome, primary snoring, infant sleep apnea, congenital central hypoventilation syndrome, sudden infant death syndrome, benign neonatal sleep myoclonus, and the like

A “sleep disorder” also arises in a subject that has other medical disorders, diseases, or injuries, or in a subject being treated with other medications or medical treatments, where the subject as a result has difficulty falling asleep and/or remaining asleep, or experiences unrefreshing sleep, e.g., the subject experiences sleep deprivation. For example, some subjects have difficulty sleeping after undergoing medical treatment for other conditions, e.g., chemotherapy or surgery, or as a result of pain or other effects of physical injuries.

It is well known in the art that certain medical disorders, for example, central nervous system (CNS) disorders, e.g. mental or neurological disorders, e.g., anxiety, can have a sleep disorder component, e.g., sleep deprivation. Thus, “treating a sleep disorder” also includes treating a sleep disorder component of other disorders, e.g., CNS disorders. Further, treating the sleep disorder component of CNS disorders can also have the beneficial effect of ameliorating other symptoms associated with the disorder. For example, in some subjects experiencing anxiety coupled with sleep deprivation, treating the sleep deprivation component also treats the anxiety component. Thus, the present invention also includes a method of treating such medical disorders.

For example, sleep disorders associated with mental disorders include psychoses, mood disorders, anxiety disorders, panic disorder, addictions, and the like. Specific mental disorders include, for example, depression, obsessive compulsive disorder, affective neurosis/disorder, depressive neurosis/disorder, anxiety neurosis; dysthymic disorder, behavior disorder, mood disorder, schizophrenia, manic depression, delirium, alcoholism, and the like.

Sleep disorders associated with neurological disorders include, for example, cerebral degenerative disorders, dementia, parkinsonism, fatal familial insomnia, sleep related epilepsy, electrical status epilepticus of sleep, sleep-related headaches, and the like. Sleep disorders associated with other medical disorders include, for example, sleeping sickness, nocturnal cardiac ischemia, chronic obstructive pulmonary disease, sleep-related asthma, sleep-related gastroesophageal reflux, peptic ulcer disease, fibrositis syndrome, and the like.

In some circumstances, sleep disorders are also associated with pain, e.g., neuropathic pain associated with restless leg syndrome; migraine; hyperalgesia, pain; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to infection, e.g., HIV, post-polio syndrome, and post-herpetic neuralgia; phantom limb pain; labor pain; cancer pain; post-chemotherapy pain; post-stroke pain; post-operative pain; neuralgia; conditions associated with visceral pain including irritable bowel syndrome, migraine and angina; and the like.

Other sleep disorders include, for example, short sleeper, long sleeper, subwakefulness syndrome, fragmentary myoclonus, sleep hyperhidrosis, menstrual-associated sleep disorder, pregnancy-associated sleep disorder, terrifying hypnagogic hallucinations, sleep-related neurogenic tachypnea, sleep-related laryngospasm, sleep choking syndrome, and the like.

Insomnia is typically classed into sleep onset insomnia, where a subject takes more than 30 minutes to fall asleep; and sleep maintenance insomnia, where the subject spends more than 30 minutes awake during an expected sleep period, or, for example, waking before the desired wake-up time with difficulty or an inability to get back to sleep. The disclosed compounds are particularly effective in treating sleep onset and sleep maintenance insomnias, insomnia resulting from circadian rhythm adjustment disorders, or insomnia resulting from CNS disorders. A preferred embodiment is treating a subject for a circadian rhythm adjustment disorder. Another preferred embodiment is treating a subject for insomnia resulting from a mood disorder. In other embodiments, a subject is treated for sleep apnea, somnambulism, night terrors, restless leg syndrome, sleep onset insomnia, and sleep maintenance insomnia; or more preferably, sleep onset insomnia or sleep maintenance insomnia. The disclosed compounds are particularly effective for treating sleep onset insomnia. The disclosed compounds are also particularly effective for treating sleep maintenance insomnia.

An “effective amount” of a compound of the disclosed invention is the quantity which, when administered to a subject in need of treatment, results in the subject falling asleep more rapidly, results in more refreshing sleep, reduces duration or frequency of waking during a sleep period, or reduces the duration, frequency, or intensity of episodes of night terrors, bruxism, or somnambulism. The amount of the disclosed compound to be administered to a subject will depend on the particular disorder, the mode of administration, co-administered compounds, if any, and the characteristics of the subject, such as general health, other diseases, age, sex, genotype, body weight and tolerance to drugs. The skilled artisan will be able to determine appropriate dosages depending on these and other factors. Effective amounts of the disclosed compounds typically range between about 0.01 mg/kg per day and about 100 mg/kg per day, and preferably between 0.1 mg/kg per day and about 10 mg/kg/day. Techniques for administration of the disclosed compounds of the invention can be found in Remington: the Science and Practice of Pharmacy, 19^{th} edition, Mack Publishing Co., Easton, Pa. (1995), the entire teachings of which are incorporated herein by reference.

The disclosed compounds may contain one or more chiral centers. The presence of chiral centers in a molecule gives rise to stereoisomers. For example, a pair of optical isomers, referred to as “enantiomers”, exist for every chiral center in a molecule. A pair of diastereomers exist for every chiral center in a compound having two or more chiral centers. Where the structural formulas or compound names do not explicitly denote stereochemistry, it is to be understood that these encompass enantiomers free from the corresponding optical isomer, racemic mixtures, mixtures enriched in one enantiomer relative to its corresponding optical isomer, a diastereomer free of other diastereomers, a pair of diastereomers free from other diasteromeric pairs, mixtures of diasteromers, mixtures of diasteromeric pairs, mixtures of diasteromers in which one diastereomer is enriched relative to the other diastereomer(s) and mixtures of diasteromeric pairs in which one diastereomeric pair is enriched relative to the other diastereomeric pair(s).

The term “stereochemically isomeric forms” as used herein defines all the possible stereoisomeric forms in which the compounds of the invention exist, including enantiomers, enantiomeric mixtures and diastereomeric mixtures. Unless otherwise mentioned or indicated, the chemical designation of compounds denotes the mixture, and in particular the racemic mixture, of all possible stereochemically isomeric forms, said mixtures containing all diastereomers and enantiomers of the basic molecular structure. Stereochemically isomeric forms of the compounds of the invention and mixtures of such forms are intended to be encompassed by the formulae used herein.

Pure stereoisomeric forms of the compounds as mentioned herein are defined as isomers substantially free of other enantiomeric or diastereomeric forms of the same basic molecular structure of the compounds. In particular, the term “stereoisomerically pure compounds” concerns compounds having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” or equivalent terms should be understood in a similar way, but then having regard to the enantiomeric excess, respectively the diastereomeric excess of the mixture in question.

A “pharmaceutically acceptable salt” of the disclosed compound is a product of the disclosed compound that contains an ionic bond, and is typically produced by reacting the disclosed compound with either an acid or a base, suitable for administering to a subject.

For example, an acid salt of a compound containing an amine or other basic group can be obtained by reacting the compound with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid.

Salts of compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, N-benzyl-∃-phenethylamine, dehydroabietylamine, N,N′-bisdehydroabietylamine, glucamine, N-methylglucamine, collidine, quinine, quinoline, and basic amino acid such as lysine and arginine.

Certain compounds and their salts may also exist in the form of solvates, for example hydrates, and the present invention includes each solvate and mixtures thereof.

As used herein, a “pharmaceutical composition” is a formulation containing the disclosed compounds in a form suitable for administration to a subject. The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of active ingredient (i.e., a formulation of the disclosed compound or salts thereof) in a unit dose of composition is an effective amount and may be varied according to the particular treatment involved. One skilled in the art will appreciate that it can be necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, and the like.

The compounds described herein, and the pharmaceutically acceptable salts thereof can be used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein. Techniques for formulation and administration of the disclosed compounds of the invention can be found in Remington: the Science and Practice of Pharmacy, above.

Typically, the compound is prepared for oral administration, wherein the disclosed compounds or salts thereof can be combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like.

For parental administration of the disclosed compounds, or salts, solvates, or hydrates thereof, can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

For rectal administration, suitable pharmaceutical compositions are, for example, topical preparations, suppositories or enemas.

In addition, the compounds may also be formulated to deliver the active agent by pulmonary administration, e.g., administration of an aerosol formulation containing the active agent from, for example, a manual pump spray, nebulizer or pressurized metered-dose inhaler. Suitable formulations of this type can also include other agents, such as antistatic agents, to maintain the disclosed compounds as effective aerosols.

The term “pulmonary” as used herein refers to any part, tissue or organ whose primary function is gas exchange with the external environment, i.e., O₂/CO₂ exchange, within a patient. “Pulmonary” typically refers to the tissues of the respiratory tract. Thus, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli). For purposes of the present invention, “pulmonary” is also meant to include a tissue or cavity that is contingent to the respiratory tract, in particular, the sinuses.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

For nasal administration, either a solid or a liquid carrier can be used. The solid carrier includes a coarse powder having particle size in the range of, for example, from about 20 to about 500 microns and such formulation is administered by rapid inhalation through the nasal passages. Where the liquid carrier is used, the formulation may be administered as a nasal spray or drops and may include oil or aqueous solutions of the active ingredients.

Also contemplated are formulations that are rapidly dispersing dosage forms, also known as “flash dose” forms. In particular, some embodiments of the present invention are formulated as compositions that release their active ingredients within a short period of time, e.g., typically less than about five minutes, preferably less than about ninety seconds, more preferably in less than about thirty seconds and most preferably in less than about ten or fifteen seconds. Such formulations are suitable for administration to a subject via a variety of routes, for example by insertion into a body cavity or application to a moist body surface or open wound.

Typically, a “flash dosage” is a solid dosage form that is administered orally, which rapidly disperses in the mouth, and hence does not require great effort in swallowing and allows the compound to be rapidly ingested or absorbed through the oral mucosal membranes. In some embodiments, suitable rapidly dispersing dosage forms are also used in other applications, including the treatment of wounds and other bodily insults and diseased states in which release of the medicament by externally supplied moisture is not possible.

“Flash dose” forms are known in the art; see for example, effervescent dosage forms and quick release coatings of insoluble microparticles in U.S. Pat. Nos. 5,578,322 and 5,607,697; freeze dried foams and liquids in U.S. Pat. Nos. 4,642,903 and 5,631,023; melt spinning of dosage forms in U.S. Pat. Nos. 4,855,326, 5,380,473 and 5,518,730; solid, free-form fabrication in U.S. Pat. No. 6,471,992; saccharide-based carrier matrix and a liquid binder in U.S. Pat. Nos. 5,587,172, 5,616,344, 6,277,406, and 5,622,719; and other forms known to the art.

Also contemplated are formulations, e.g., liquid formulations, including cyclic or acyclic encapsulating or solvating agents, e.g., cyclodextrins, polyethers, or polysaccharides (e.g., methylcellulose), or more preferably, polyanionic β-cyclodextrin derivatives with a sodium sulfonate salt group separate from the lipophilic cavity by an alkyl ether spacer group or polysaccharides. In a preferred embodiment, the agent is methylcellulose. In another preferred embodiment, the agent is a polyanionic β-cyclodextrin derivative with a sodium sulfonate salt separated from the lipophilic cavity by a butyl ether spacer group, e.g.) CAPTISOL® (CyDex, Overland, Kans.). One skilled in the art can evaluate suitable agent/disclosed compound formulation ratios by preparing a solution of the agent in water, e.g., a 40% by weight solution; preparing serial dilutions, e.g. to make solutions of 20%, 10, 5%, 2.5%, 0% (control), and the like; adding an excess (compared to the amount that can be solubilized by the agent) of the disclosed compound; mixing under appropriate conditions, e.g., heating, agitation, sonication, and the like; centrifuging or filtering the resulting mixtures to obtain clear solutions; and analyzing the solutions for concentration of the disclosed compound.

In addition to the therapeutic formulations described above, a therapy including the compounds of the present invention optionally includes, or be co-administered with one or more additional therapies, e.g., drugs or physical or behavioral treatments (e.g., light therapy, electrical stimulation, behavior modification, cognitive therapy, circadian rhythm modification, and the like). Such a practice is referred to as “combination therapy.” The other therapy or therapies in the combination therapy include therapies recognized by one skilled in the art as desirable in combination with the compound of the invention, for example, therapies known to the art or therapies which are proposed or discovered in the art for treating sleep disorders or treating diseases associated with sleep disorders, for example, therapies for any of the sleep disorders or other conditions disclosed herein. In some embodiments the compound is administered as a combination therapy whereas it is administered as a monotherapy in other embodiments. Typically, the compound is administered as a monotherapy.

It will be appreciated by one skilled in the art that a therapy administered in combination with the compounds of the present invention can be directed to the same or a different disorder target as that being targeted by the compounds of the present invention. Administration of the compound of the invention may be first, followed by the other therapy; or administration of the other therapy may be first. The other therapy is any known in the art to treat, prevent, or reduce the symptoms of the targeted disorder, e.g., a sleep disorder, or other disorders, e.g., other CNS disorders. In addition, some embodiments of the present invention have compounds administered in combination with other known therapies for the target disorder. Furthermore, the other therapy includes any agent of benefit to the patient when administered in combination with the disclosed compound.

For example, in some embodiments where the other therapy is a drug, it is administered as a separate formulation or in the same formulation as the compound of the invention. A compound of the invention is administered in combination therapy with any one or more of commercially-available, over-the-counter or prescription medications, including, but not limited to antimicrobial agents, fungistatic agents, germicidal agents, hormones, antipyretic agents, antidiabetic agents, bronchodilators, antidiarrheal agents, antiarrhythmic agents, coronary dilation agents, glycosides, spasmolytics, antihypertensive agents, antidepressants, antianxiety agents, other psychotherapeutic agents, corticosteroids, analgesics, contraceptives, nonsteroidal anti-inflammatory drugs, blood glucose lowering agents, cholesterol lowering agents, anticonvulsant agents, other antiepileptic agents, immunomodulators, anticholinergics, sympatholytics, sympathomimetics, vasodilatory agents, anticoagulants, antiarrhythmics, prostaglandins having various pharmacologic activities, diuretics, sleep aids, antihistaminic agents, antineoplastic agents, oncolytic agents, antiandrogens, antimalarial agents, antileprosy agents, and various other types of drugs. See Goodman and Gilman's The Basis of Therapeutics (Eighth Edition, Pergamon Press, Inc., USA, 1990) and The Merck Index (Eleventh Edition, Merck & Co., Inc., USA, 1989).

In addition to the formulations described above, a formulation can optionally include, or be co-administered with sedatives, vitamins, antihistamines, steroids, and the like. Typically, the compound is administered as a monotherapy.

The term “derivative”, e.g., in the term “N-substituted azaheterocyclic compound derivatives”, refers to compounds that have a common core structure, and are substituted with various groups as described herein. For example, all of the compounds represented by structural formula I are N-substituted azaheterocyclic derivatives, and have structural formula I as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include acyl sulfonimides, tetrazoles, sulfonates, and phosphonates. See, e.g., Patani and LaVoie, Chem. Rev. 96, 3147-3176 (1996).

The term “C₁₋₆-alkyl” as used herein, alone or in combination, refers to a straight or branched, saturated hydrocarbon chain having 1 to 6 carbon atoms such as e.g. methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-hexyl, 1,2-dimethylpropyl, 2,2-dimethylpropyl and 1,2,2-trimethylpropyl, and the like.

The term “C₁₋₆alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms e.g. methoxy, ethoxy, propoxy, isopropoxy, butoxy, pentoxy, and the like.

The term “halogen” means fluorine, chlorine, bromine or iodine.

The term “nonaromatic nitrogen containing heterocycle” (e.g. the nonaromatic heterocyclic groups represented by Z, e.g., in Z^{a-l}) refers to non-aromatic ring systems typically having three to eight members, preferably three to six, in which at least one ring carbon is replaced with a nitrogen and zero, one or more additional ring carbons, preferably zero to one, are each replaced by a heteroatom such as N, O, or S. Examples of nonaromatic nitrogen containing heterocycles include aziridine, pyrrolidine, piperidine, piperazine, morpholine, tetrahydroquinoline, tetrahydroisoquinoline, thiomorpholine, thiazolidine, and the like. “Attached through a ring nitrogen” means the group is bonded to the rest of the molecule through a ring nitrogen.

The term “aryl” group, (e.g., the aryl groups represented by Z, e.g., in Z^{e}, Z^{f}, and Zⁿ) refers to carbocyclic aromatic groups such as phenyl, naphthyl, and anthracyl, or groups in which a phenyl group is fused to another ring, for example, the group-s represented by Z^{e} and Z^{f}. As used herein, a “heteroaryl” group is a 5 membered carbocyclic ring containing at least one N, S, or O atom and two double bonds, or a 6 membered carbocyclic ring containing at least one N, S, or O atom and three double bonds. Examples of “heteroaryl” groups (e.g., the heteroaryl group represented by Z, e.g., in Z^{m}) refers to heteroaromatic groups such as imidazolyl, isoimidazolyl, thienyl, furanyl, pyridyl, pyrimidyl, pyranyl, pyrazolyl, pyrrolyl, pyrazinyl, thiazolyl, isothiazolyl, oxazolyl, isooxazolyl, 1,2,3-triazolyl, 1,2,4-triazolyl, tetrazolyl, and the like.

The term “cycloalkyl group” (e.g., the cycloalkyl groups represented by Z, e.g. in Z^{o}) is a cyclic alkyl group has from 3 to about 10 carbon atoms, preferably from 3 to about 7. Examples of suitable cycloalkyl groups include cyclopropyl, cyclobutyl, cyclopentyl, cyclohexyl, cycloheptyl and cyclooctyl.

The term “aliphatic” (e.g., the aliphatic groups represented by B) includes branched and linear alkyl groups that contain zero, one or more units of carbon-carbon unsaturation, i.e., carbon-carbon double or triple bonds. A cycloaliphatic group is a cyclic aliphatic group, for example, cyclohexenyl or cyclopentenyl.

The terms “aralkyl” and “heteroaralkyl” refer to aryl and heteroaryl, groups, respectively, that are connected through an alkyl chain, e.g., benzyl, ethylpyridine, and the like.

Suitable substituents for aryl aralkyl, heteroaryl, heteroaralkyl, nonaromatic heterocyclyl, alkyl, cycloalkyl, aliphatic, and cycloaliphatic groups are those that do not substantially interfere with the pharmaceutical activity of the disclosed compound. A group can have one or more substituents, which can be identical or different. Examples of suitable substituents for a substitutable carbon atom in an alkyl, aliphatic, cycloalkyl, cycloaliphatic, non-aromatic heterocyclic, aryl, or heteroaryl group include —OH, halogen (—Br, —Cl, —I and —F), —R, —OR, —CH₂R, —CH₂CH₂R, —OCH₂R, —CH₂OR, —CH₂CH₂OR, —CH₂OC(O)R, —O—COR, —COR, —SR, —SCH₂R, —CH₂SR, —SOR, —SO₂R, —CN, —NO₂, —COOH, —SO₃H, —NH₂, —NHR, —N(R)₂, —COOR, —CH₂COOR, —CH₂CH₂COOR, —CHO, —CONH₂, —CONHR, —CON(R)₂, —NHCOR, —NRCOR, —NHCONH₂, —NHCONRH, —NHCON(R)₂, —NRCONH₂, —NRCONRH, —NRCON(R)₂, —C(═NH)—NH₂, —C(═NH)—NHR, —C(═NH)—N(R)₂, —C(═NR)—NH₂, —C(═NR)—NHR, —C(═NR)—N(R)₂, —NH—C(═NH)—NH₂, —NH—C(═NH)—NHR, —NH—C(═NH)—N(R)₂, —NH—C(═NR)—NH₂, —NH—C(═NR)—NHR, —NH—C(═NR)—N(R)₂, —NRH—C(═NH)—NH₂, —NR—C(═NH)—NHR, —NR—C(═NH)—N(R)₂, —NR—C(═NR)—NH₂, —NR—C(═NR)—NHR, —NR—C(═NR)—N(R)₂, —SO₂NH₂, —SO₂NHR, —SO₂NR₂, —SH, —SOₖR (k is 0, 1 or 2) and —NH—C(═NH)—NH₂. Each R is independently an alkyl, cycloalkyl, benzyl, aromatic, heteroaromatic, or phenylamine group that is optionally substituted. Preferably, R is unsubstituted. In addition, —N(R)₂, taken together, can also form a substituted or unsubstituted heterocyclic group, such as pyrrolidinyl, piperidinyl, morpholinyl and thiomorpholinyl. Examples of substituents on group represented by R include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl.

Suitable substituents on the nitrogen of a heterocyclic group or heteroaromatic group include —R′, —N(R′)₂, —C(O)R′, —CO₂R′, —C(O)C(O)R′, —C(O)CH₂C(O)R′, —SO₂R′, —SO₂N(R′)₂, —C(═S)N(R′)₂, —C(═NH)—N(R′)₂, and —NR′SO₂R′. R′ is —H, an alkyl, alkoxy, cycloalkyl, cycloalkoxy, phenyl, phenoxy, benzyl, benzyloxy, heteroaromatic, or heterocyclic group that is optionally substituted. Examples of substituents on the groups represented by R′ include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl. Preferably, R′ is unsubstituted.

Some compounds described herein may be synthesized by methods known to one skilled in the art. Detailed methods are described in U.S. Pat. No. 6,569,849, U.S. Pat. No. 6,239,148, and U.S. Pat. No. 5,712,292, the entire teachings of each are incorporated herein by reference.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLE 1

### Disclosed Compounds are Effective Sleep-Inducing Agents

Sleep in mammals can be divided into sleep occurring during periods of rapid eye movement (REM), accompanied by substantial brain activity, and periods of non-REM (NREM) sleep, accompanied by decreased brain activity. Typically, a normal nighttime sleep period is occupied primarily by NREM sleep, and thus NREM cumulation can serve as a measure of total sleep cumulation, i.e., significantly decreased NREM can be associated with insomnia and an accumulation of “sleep debt”, e.g. an accumulated physiological need for sleep that tends to persist until a sufficient amount of additional sleep is accumulated. Thus, an increase in NREM associated with a treatment can indicated the treatment's effectiveness in treating insomnia.

Sleep quality can be associated with sleep continuity or sleep maintenance. For example, a subject with sleep apnea wakes up numerous times during a sleep period, e.g., the subject has difficulty maintaining continuous sleep. Although such a subject can accumulate a typical nights length of sleep, e.g., 8 hours, the sleep is unrefreshing due to the waking caused by the sleep apnea. Thus, an increase in longest uninterrupted sleep bout (LUSB) associated with a treatment can indicated the treatment's effectiveness in treating sleep maintenance insomnia.

Sleep-wakefulness, locomotor activity and body temperature were monitored in vivo with the disclosed racemic sleep-inducing agent 1 and its R and S isomers 1-R and 1-S. Adult, male Wistar rats (250 g at time of surgery, Charles River Laboratories, Wilmington Mass.) were anesthetized (Nembutal, 62 mg/kg) and surgically prepared with a cranial implant to permit chronic electro-encephalogram (EEG) and electromyogram (EMG) recording. Body temperature and locomotor activity were monitored via a miniature transmitter (Mini-Mitter, Bend, Oreg.) surgically placed in the abdomen. The cranial implant consisted of stainless steel screws (two frontal [+3.2 AP from bregma, ±2.0 ML] and two occipital [−6.9 AP, ±5.5 ML]) for EEG recording. Two Teflon®-coated stainless steel wires were positioned under the nuchal trapezoid muscles for EMG recording. All leads were soldered to a miniature connector prior to surgery, and gas sterilized in ethylene oxide. The implant assembly was affixed to the skull with dental acrylic. A minimum of three weeks was allowed for surgical recovery.

Each rat was permanently housed in its own individual recording cage located within separate, ventilated compartments of custom- designed stainless steel cabinets. Each cage was enhanced with a filter-top riser and low-torque swivel-commutator. Food and water were available ad libitum. A 24-hr light-dark cycle (12 hours light, 12 hours dark) was maintained throughout the study using 4-watt fluorescent bulbs 5 cm from the cage. Animals were undisturbed for at least 48 hours before and after treatments.

Sleep and wakefulness were determined using “SCORE-2000™” (Hypnion, Worcester, Mass.)—an internet-based sleep-wake and physiological monitoring system. The system monitored amplified EEG (bandpass 1-30 Hz; digitization rate 400 Hz), integrated EMG (bandpass 10-100 Hz), body temperature and non-specific locomotor activity (LMA) via telemetry, and drinking activity, continuously and simultaneously. Arousal states were classified on-line as non-REM (NREM) sleep, REM sleep, wake, or theta-dominated wake every 10 seconds using EEG feature extraction and pattern-matching algorithms. From this data, the longest uninterrupted sleep bout (LUSB) could be obtained. The classification algorithm used individually-taught EEG-arousal-state templates, plus EMG criteria to differentiate REM sleep from theta-dominated wakefulness, plus behavior-dependent contextual rules (e.g., if the animal was drinking, it is awake). Drinking and locomotor activity (LMA) were recorded as discrete events every 10 seconds, while body temperature was recorded each minute. Locomotor activity was detected by a telemetry receiver (Mini-Mitter) beneath the cage. Telemetry measures (LMA and body temperature) were not part of the scoring algorithm; thus, sleep-scoring and telemetry data were independent measures.

Compounds were administered at CT-18, the peak of the activity-dominated period, in order to ensure prior wakefulness was sufficient to interact positively with hypnotic-drug effects, and sufficient time was allowed to view the time course of the treatment effect before lights-on (6 hours post-treatment). Compounds were suspended in sterile 0.25% or 0.5% methylcellulose (2 ml/kg). Treatments were administered as an intraperitoneal bolus.

A parallel group study design was employed. Vehicle controls were drawn from a large pool (N>200): a subset of the pooled vehicle controls was selected, based on computerized matching with the 24-hour pre-treatment baseline of the active treatment group.

The results of NREM and LUSB parameters were measured for racemic sleep-inducing agent 1 and its R and S isomers 1-R and 1-R. (Table 1).

TABLE 1
Disclosed Compounds Are Effective Sleep Inducing Agents

| Compound | Dose, mg/kg | N | NREM | LUSB |
|---|---|---|---|---|
| 1 (Racemate) | 3 | 14 | 15 | 4.7 |
| | 10 | 11 | 24.8 | 2.8 |
| | 30 | 15 | 35.4 | 4.9 |
| 1-R | 10 | 9 | 3.4 | 2.5 |
| | 30 | 12 | 20.7 | 5.8 |
| | 45 | 4 | 31.8 | 10.3 |
| 1-S | 10 | 9 | 31.9 | 5.5 |
| | 30 | 10 | 31.8 | 9.7 |

In Table 1, N is the number of trials, and NREM and LUSB values are means adjusted for both baseline and vehicle control values. NREM represents the mean maximum cumulation of NREM sleep in minutes over baseline as a function of time in hours after treatment with the compound or the vehicle control; LUSB is the longest uninterrupted sleep bout each hour in minutes as a function of circadian time.

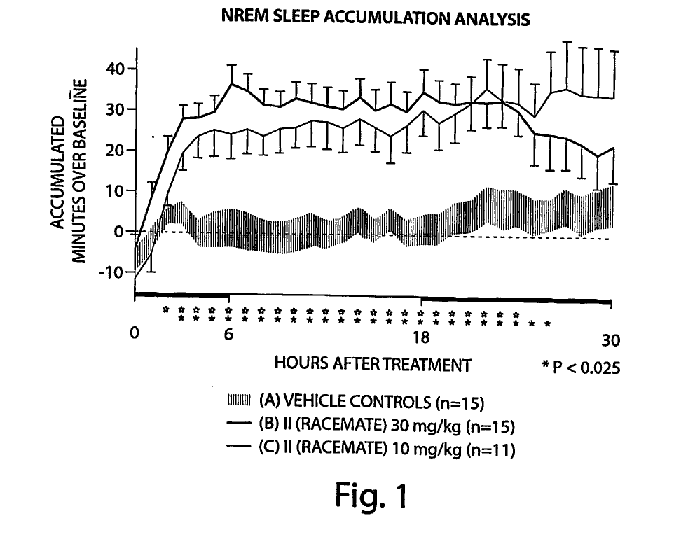

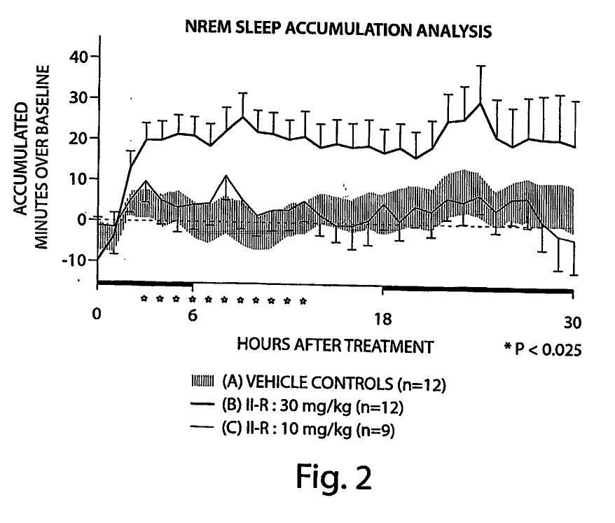

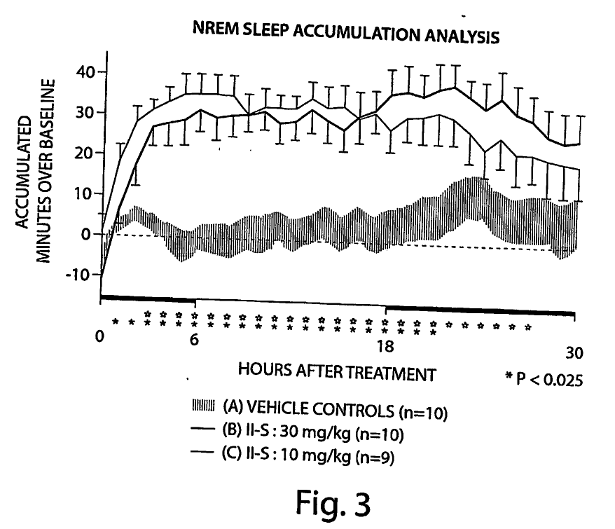

FIGS. 1, 2, and 3, respectively, show NREM sleep cumulation for racemate 1, 1-R and 1-S, respectively, at 10 mg/kg (grey line) and 30 mg/kg (black line) versus vehicle control (grey band). As can be seen, each of the three compounds is effective at increasing NREM cumulation at a dose of 30 mg/kg, with racemic 1 and 1-S being more effective than 1-R. At a dose of 10 mg/kg, racemic 1 and 1-S are particularly effective. Thus, all three compounds can be effective at decreasing insomnia by increasing cumulative NREM time.

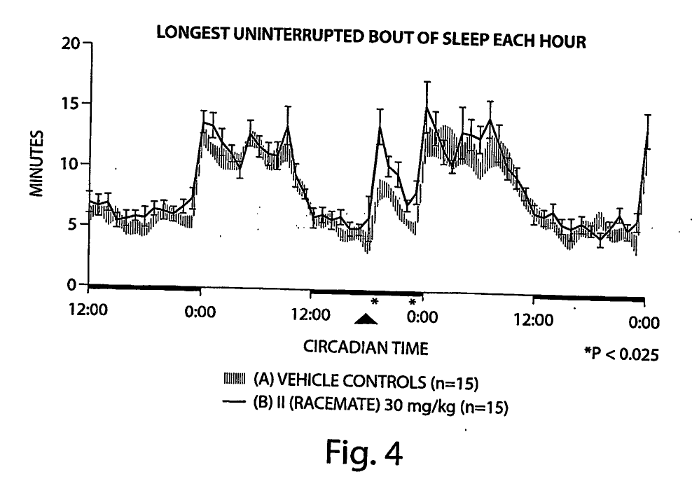

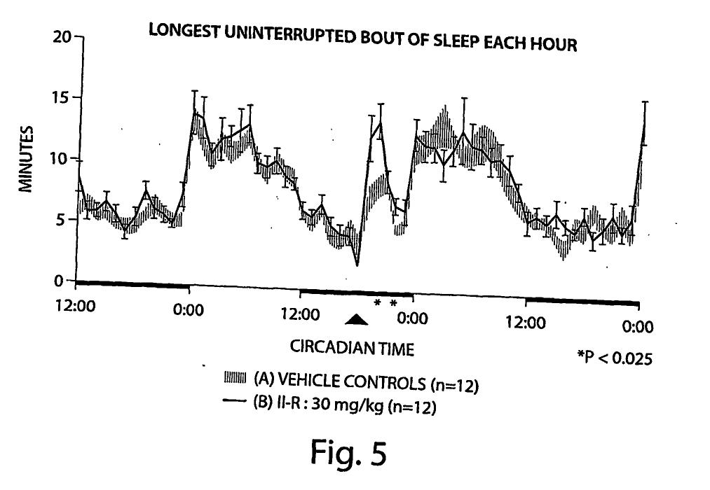

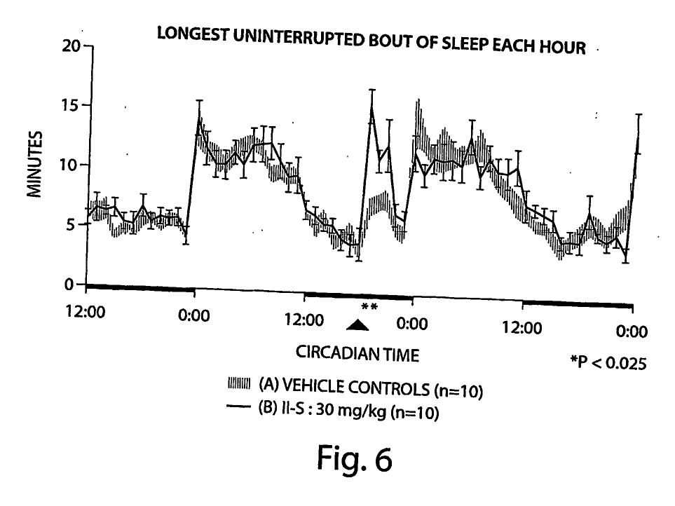

FIGS. 4, 5, and 6, show the longest uninterrupted sleep bouts (LUSB) for racemate 1, 1-R and 1-S, respectively, at 30 mg/kg (black line) versus vehicle control (grey band). As can be seen, at a dose of 30 mg/kg, each of the three compounds is effective at increasing LUSB, in some cases to as much as 15 minutes compared to about 10 minutes with the vehicle control. Thus, because all three compounds increase LUSB time, they will be effective for decreasing sleep-maintenance insomnia.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a subject for a sleep disorder, comprising administering to a subject in need of treatment for a sleeping disorder an effective amount of a compound represented by structural formula II; 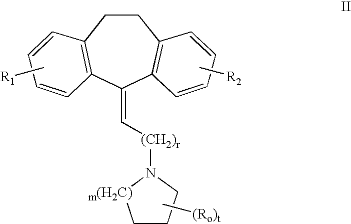
wherein
R¹ and R² independently are —H, halogen, hydroxy, —CN, —NO₂, C₁₋₆-alkyl, halogenated C₁₋₆-alkyl, C₁₋₆-alkoxy, halogenated C₁₋₆-alkoxy, —NR²¹R²², —(SO₂)NR²¹R²², —NR²¹(SO₂)NR²², —(CO)NR²¹R²², —NR²¹(CO)R²², —(CO)R²², or —(CO₂)R²²,
R²¹ and R²² independently are —H or C₁₋₆-alkyl;
Rₒ is C₁₋₆-alkyl, halogenated C₁₋₆-alkyl, C₁₋₆-alkoxy, halogenated C₁₋₆-alkoxy, phenyl, phenyl substituted with halogen, hydroxy, nitro or cyano, —(CH₂)_{q}COR^{p}, —(CH₂)_{q}CONHSO₂Aryl, —(CH₂)_{q}CONHSO₂Heteroaryl, —(CH₂)_{q}CONHS(O)₂-Alkyl, —(CH₂)_{q}OH—(CH₂)_{q}SO₂R^{p}, —(CH₂)_{q}S(O)₂NHCO-alkyl, —(CH₂)_{q}S(O)₂NHCO-aryl, —(CH₂)_{q}S(O)NHCO-alkyl, —(CH₂)_{q}S(O)NHCO-aryl, —(CH₂)_{q}P(O)(OH)₂, —(CH₂)_{q}P(O)OH, 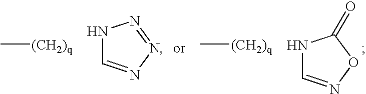
wherein q is 0, 1 or 2;
R^{p} is H, OH or C₁₋₈ alkyl,
r is 1, 2, 3 or 4;
m is 1 or 2, and
t is 1, 2, or 3; or a pharmaceutically acceptable salt, solvate, or hydrate thereof.

2. The method of claim 1 wherein the subject is a human.

3. The method of claim 1, wherein the subject is treated for a circadian rhythm adjustment disorder.

4. The method of claim 1, wherein the subject is treated for a sleep disorder selected from sleep apnea, somnambulism, night terrors, restless leg syndrome, sleep onset insomnia, and sleep maintenance insomnia.

5. The method of claim 4 wherein the subject is treated for sleep onset insomnia or sleep maintenance insomnia.

6. A method of treating a subject for a sleep disorder, comprising administering to a subject in need of treatment for a sleeping disorder an effective amount of a compound represented by structural formula IIIa or IIIb: 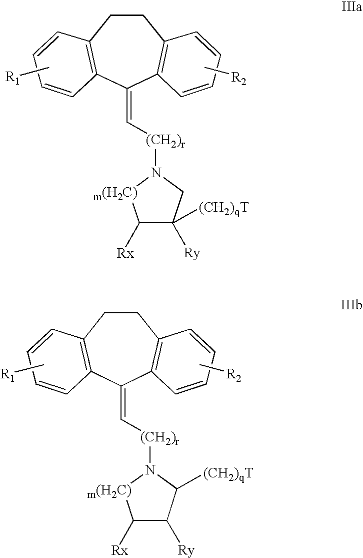
wherein
R¹ and R² independently are —H, halogen, hydroxy, —CN, —NO₂, C₁₋₆-alkyl, halogenated C₁₋₆-alkyl, C₁₋₆-alkoxy, halogenated C₁₋₆-alkoxy, —NR²¹R², —(SO₂)NR²¹R²², —NR²¹(SO₂)NR²², —(CO)NR²¹R²², —NR²¹(CO)R²², —(CO)R²², or —(CO₂)R²², and R²¹ and R²² independently are —H or C₁₋₆-alkyl;
Rₓ and R_{y} are, independently, hydrogen, C₁₋₆-alkyl, halogenated C₁₋₆-alkyl, C₁₋₆-alkoxy, halogenated C₁₋₆-alkoxy, or, taken together, Rₓ and R_{y} form a bond;
T is COOH, COOR^{a}, CONHSO₂Aryl, CONHSO₂Heteroaryl, CONHS(O)₂Alkyl, SO₃H, S(O)₂NHCOAlkyl, S(O)₂NHCOAryl, S(O)NHCOAlkyl, (O)NHCO-Aryl, P(O)(OH)₂, 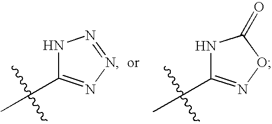
R^{a} is C₁-C₆ alkyl;
q is 0, 1 or 2;
r is 1, 2, 3 or 4; and
m is 1 or 2; or a pharmaceutically acceptable salt, solvate, or hydrate thereof.

7. The method of claim 6 wherein the subject is a human.

8. The method of claim 6, wherein the subject is treated for a circadian rhythm adjustment disorder.

9. The method of claim 6, wherein the subject is treated for a sleep disorder selected from sleep apnea, somnambulism, night terrors, restless leg syndrome, sleep onset insomnia, and sleep maintenance insomnia.

10. The method of claim 9 wherein the subject is treated for sleep onset insomnia or sleep maintenance insomnia.

11. A method of treating a subject for a sleep disorder, comprising administering to a subject in need of treatment for a sleeping disorder an effective amount of a compound represented by structural formula IVa or IVb: 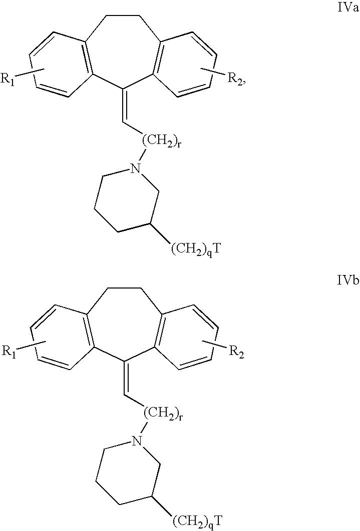
wherein
R¹ and R² independently are —H, halogen, hydroxy, —CN, —NO₂, C₁₋₆-alkyl, halogenated C₁₋₆-alkyl, C₁₋₆-alkoxy, halogenated C₁₋₆-alkoxy, —NR²¹R²², —(SO₂)NR²¹R²², —NR²l(SO₂)NR²², —(CO)NR²¹R²², —NR²¹(CO)R²², —(CO)R²², or —(CO₂)R²², and R²¹ and R²² independently are —H or C₁₋₆-alkyl;
T is COOH, COOR^{a}, CONHSO₂Aryl, CONHSO₂Heteroaryl, CONHS(O)₂Alkyl, SO₃H, S(O)₂NHCOAlkyl, S(O)₂NHCOAryl, S(O)NHCOAlkyl, S(O)NHCO-Aryl, P(O)(OH)₂, 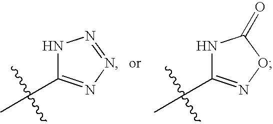
R^{a} is C₁-C₆ alkyl;
q is 0, 1 or 2; and
r is 1, 2, 3 or 4; or a pharmaceutically acceptable salt, solvate, or hydrate thereof.

12. The method of claim 11 wherein the subject is a human.

13. The method of claim 11, wherein the subject is treated for a circadian rhythm adjustment disorder.

14. The method of claim 11, wherein the subject is treated for a sleep disorder selected from sleep apnea, somnambulism, night terrors, restless leg syndrome, sleep onset insomnia, and sleep maintenance insomnia.

15. The method of claim 14 wherein the subject is treated for sleep onset insomnia or sleep maintenance insomnia.

16. A method of treating a subject for a sleep disorder, comprising administering to a subject in need of treatment for a sleeping disorder an effective amount of a compound represented by structural formula Va or Vb: 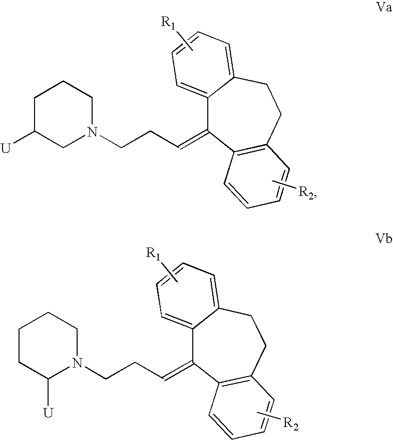
wherein
R¹ and R² independently are —H, halogen, hydroxy, —CN, —NO₂, o C₁₋₆-alkyl, halogenated C₁₋₆-allyl, C₁₋₆-alkoxy, halogenated C₁₋₆-alkoxy, —NR²¹R²², —(SO₂)NR²¹R²², —NR²¹(SO₂)NR₂₂, —(CO)NR²¹R²², —NR²¹(CO)R²², —(CO)R²², or —(CO₂)R²², R²¹ and R²² independently are —H or C₁₋₆-alkyl;
R^{a} is C₁-C₆ alkyl; and
U is COOH, COOR^{a}, CONHSO₂Aryl, 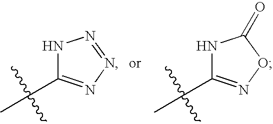
or a pharmaceutically acceptable salt, solvate, or hydrate thereof.

17. The method of claim 16 wherein the subject is a human.

18. The method of claim 16, wherein the subject is treated for a circadian rhythm adjustment disorder.

19. The method of claim 16, wherein the subject is treated for a sleep disorder selected from sleep apnea, somnambulism, night terrors, restless leg syndrome, sleep onset insomnia, and sleep maintenance insomnia.

20. The method of claim 19 wherein the subject is treated for sleep onset insomnia or sleep maintenance insomnia.

21. The method of claim 1, 6, 11, or 16 wherein R¹ and R² independently are —H, halogen, hydroxy, C₁₋₆-alkyl or C₁₋₆-alkoxy.

22. The method of claim 1, 6, 11, or 16, wherein R¹ and R² independently are —H or halogen.

23. A method of treating a subject for insomnia, comprising administering to a subject in need of treatment for insomnia an effective amount of a compound 1: 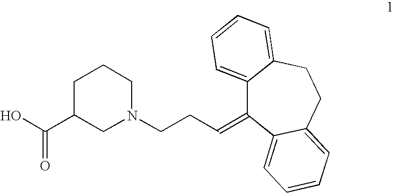
or a pharmaceutically acceptable salt, solvate, or hydrate thereof.

24. The method of claim 23, wherein the compound is the R isomer of compound 1.

25. The method of claim 23, wherein the compound is the S isomer of compound 1.
